(19) United States
(12) Patent Application Publication
(10) Pub. No.: US 2025/0025489 A1
(43) Pub. Date: Jan. 23, 2025

(54) METHODS OF VACCINATION IN PREMALIGNANT SETTINGS

(71) Applicant: University of Miami, Miami, FL (US)

(72) Inventors: Eli GILBOA, Miami, FL (US); Greta GARRIDO, Miami, FL (US); Brett SCHRAND, Miami, FL (US); Agata LEVAY, Miami, FL (US)

(21) Appl. No.: 18/906,621

(22) Filed: Oct. 4, 2024

Related U.S. Application Data
(63) Continuation of application No. 16/619,656, filed on Dec. 5, 2019.
(60) Provisional application No. 62/517,610, filed on Jun. 9, 2017.

(51) Int. Cl. A61K 31/713; A61K 39/00

(52) U.S. Cl. CPC A61K 31/713; A61K 39/4615; A61K 39/4622; A61K 39/464417; A61K 2039/585

(57) ABSTRACT

The present invention relates, in part, to methods of generating immune responses in subjects that have a likelihood of developing cancer.

### RELATED APPLICATIONS

This application claims the benefit of U.S. Provisional Application No. 62/517,610, filed on Jun. 9, 2017, the contents of which is incorporated herein by reference in its entirety.

### INCORPORATION OF SEQUENCE LISTING

The contents of the text file named “SEB-004PC-UMIP-95/118595_ST25”, which was created on May 15, 2018 and is 1 KB in size, are hereby incorporated by reference in their entirety.

### FIELD OF THE INVENTION

The present invention relates, in part, to methods for generating immune responses that reduce the likelihood of cancer onset, progression or recurrence.

### BACKGROUND

According to the World Health Organization, cancer is a global pandemic that causes nearly 7 million deaths each year worldwide. That number is expected to reach 10 million by the year 2020.

In some cases, the cancer patients have had solid tumor mass removed by surgery. However, surgical techniques often fail to remove all traces of cancer and, accordingly, reemergence of cancer is possible. Likewise, while chemotherapeutic and radiation techniques may lead to remission, there remains a likelihood that cancer may return. Tumor recurrence remains a major challenge in cancer therapy, and individuals with premalignant lesions, chronic infections, or genetic predisposition, are at high risk of developing cancer. Given the long often unpredictable time to tumor recurrence or progression of precancerous lesions to malignant tumors, the development of therapeutic strategies to prevent recurrence in cancer patients or tumor progression in at risk individuals has been challenging, representing an important unmet medical need.

Further, even subjects that have no history of cancer may be particularly susceptible to the disease. For instance, factors such as family history, genetic predisposition, occupation, and exposure to certain agents make some people are statistically more prone to develop cancer than others.

There is a need for treating subjects to reduce or prevent the onset, progression or recurrence of cancers.

### SUMMARY OF THE INVENTION

Accordingly, the present invention provides methods of altering the immune system of a subject that is susceptible to having cancer. For instance, the present methods may stimulate an immune response, e.g., a vaccine response, against future tumors. In various embodiments, the present methods induce neoantigens which are not present in a patient's future tumor and, accordingly, a patient's immune response can be directed to the tumor when it develops (e.g., by inducing these neoantigens in the tumor).

In various embodiments, the present methods allow for vaccinating against neoantigens that are not expressed in a tumor that may develop in a patient in the future and, if or when the tumor recurs and/or develops, inducing the same neoantigens in the said tumors. In this way, a patient's pre-existing immune response against neoantigens can be harnessed against an eventual tumor in a directed way (i.e. by causing the tumor to prompt an immune response to the neoantigens).

In various embodiments, the present methods reduce the likelihood of cancer onset, progression or recurrence.

In various embodiments, the present invention provides a method of treating cancer in a subject need thereof, by administering an effective amount of an immune-modulating agent to the subject's cancer cells to direct a subject's existing immune response to a neoantigen against the cancer, where the immune-modulating agent inhibits and/or downregulates a mediator of antigen processing and induces neoantigen formation; and the subject has an existing immune response against the induced neoantigen.

In various embodiments, the present invention provides a method of treating cancer by vaccinating a subject in need with an immune modulatory agent to stimulate neoantigen-directed immune response in the patient and upon tumor development, treating the tumor with immune modulatory agent(s) to stimulate (the same) neoantigens in the tumor and direct the pre-existing immune response against the tumor.

In various embodiments, pre- and post-tumor, the immune modulatory agent(s) may be the same or different. In various embodiments, pre- and post-tumor, the neoantigens stimulated are the same.

The present methods are particularly useful in subjects that are in cancer remission (e.g., have previously been afflicted with a cancer, e.g., having minimal residual disease (MRD)) and/or are predisposed to cancer (e.g., by having one or more risk factors for cancer).

Illustrative embodiments are depicted in FIG. 1.

### BRIEF DESCRIPTION OF THE DRAWINGS

FIG. 1 shows an embodiment of the invention, i.e. a non-limiting paradigm of vaccinating against neoantigens that will be induced in a future tumor. Patients in remission (A) or individuals at high risk are (B) are vaccinated against neoantigens (dots), and if or when a tumor arises the same antigens (dots) are induced in the tumor (e.g., by inhibiting and/or downregulating one or more of TAP, ERAAP, and Ii (or less favorably NMD), as discussed herein).

FIG. 2 shows tumor targeted inhibition of ERAAP or TAP inhibits tumor growth and potentiates PD-1 blockade. The nucleolin aptamer-Smg-1, ERAAP or TAP siRNA conjugates were constructed and characterized as described in Nature. 2010; 465(227-31). Balb/c mice were injected subcutaneously with 4T1 breast carcinoma tumor cells and when tumors became palpable (day 8-10), mice were injected intravenously with Nucl-siRNA conjugate or intraperitoneally with anti-PD-1 antibody as indicated. Treatments were repeated three times (left) or twice (right), 3 days apart. (*P<0.05).

FIG. 3 shows nucleolin targeted NMD and ERAAP downregulation inhibits tumor growth in the BRAF/PTEN model. 21 days old F1 BRAF/PTEN mice were treated with hydroxytamoxifen on the back and 26 days later when tumors reached an approximate height of 2-3 mm, mice were injected intravenously with Smg-1 (NMD), ERAAP or scrambled (Scram) siRNAs conjugated to nucleolin aptamer (Nuc) conjugate or control siRNA conjugates three times 3 days apart. Mice were sacrificed when tumors reached a diameter of 12 mm or when they became ulcerated.

FIG. 4 shows prophylactic cancer vaccination against NMD inhibition induced neoantigens. CT26 expressing Smg-1 shRNA were treated or not with DOX for 24 hours, irradiated and injected subcutaneously into Balb/c mice. Vaccination was repeated 7 days later. 21 days after the second vaccination mice were challenged with 4T1 tumor cells. When tumors became palpable (7-8 days later) mice were injected intravenously via the tail vein with nucleolin aptamer-Smg-1 or control siRNA conjugates as indicated and 4T1 tumor growth was monitored.

FIG. 5 shows biodistribution of Nuc aptamer-TAP siRNA conjugate in 4T1 tumor bearing mice. Balb/c mice were implanted subcutaneously with 4T1 tumor cells and when tumors reached a diameter of ˜3 mm ³²P-labeled Nuc aptamer-TAP siRNA conjugate was injected via the tail vein. 18 hours later mice were sacrificed, organs were isolated, partially perfused by incubation for 30 min in PBS at room temperature, and radioactivity counted.

FIG. 6 shows tumor infiltration of immune cell subsets. Subcutaneously implanted palpable 4T1 tumor bearing mice were treated three times with Nucl-TAP siRNA conjugates 3 days apart, and 2 days after the last treatment tumors were excised homogenized and analyzed for immune cell subsets by flow cytometry gated on CD45+CD3+ cells using the CytoFLex and Kaluza software (Beckman Coulter). Data are presented as percent of total cells except for Treg presented as percent of CD45+CD3+CD4+ cells.

FIG. 7 shows that inhibition of NMD enhances the anchorage independent growth of CT26 tumor cells. CT26 stably expressing Smg-1 siRNA under doxcycyline control (see Nature. 2010; 465(227-31) were plated in soft agar, 104 cells per 1 cm plate and were grown under either normoxic conditions (20% O₂) or hypoxic conditions (one week in 0.5% O₂ followed by one week of 20% O₂). After two weeks' colonies were counted. For each histogram, the left bar is without doxycyline and the right bar is with doxycyline.

FIG. 8 shows that tumor targeted invariant chain (li) downregulation inhibits tumor growth and enhances PD-1 blockade. Balb/c mice were implanted with 4T1 breast carcinoma cells subcutaneously and when tumors became palpable, around day 9, mice were treated systemically by intraveneous administration of nucleolin aptamer conjugated to an invariant chain or scrambled siRNA twice 3 days apart (top panel). As indicated, PD-1 Ab was administered intraperitoneally one day after aptamer-siRNA conjugate administration three times 3 days apart. Tumor growth was determined by measuring tumor volume (bottom left panel) or monitoring for tumor regression (bottom right panel, showing from top to bottom at the final time point: nucleolin aptamer conjugated to an invariant chain combined with an anti-PD-1 Ab; nucleolin aptamer conjugated to scrambled siRNA combined with an anti-PD-1 Ab; an anti-PD-1 Ab, nucleolin aptamer conjugated to an invariant chain, nucleolin aptamer conjugated to scrambled siRNA and untreated).

FIG. 9 shows prophylactic vaccination against future tumors with CpG-TAP siRNAs. CpG oligonucleotide (Nat Biotechnol 27: 925-932) extended with a short sequence was hybridized to its complementary sequence engineered at the 5′ end of the sense strand of a TAP siRNA or control siRNA (Scram). The CpG-TAP siRNA was administered three times weekly by tail vein injection into Balb/c mice. 4 weeks after last injection mice were challenged subcutaneously with 4T1 breast carcinoma cells and 7-8 days later when tumors became palpable, mice were administered by tail vein injection with nucleolin aptamer-TAP siRNA (Nuc-TAP) or control siRNA (nuc-Scram) (see top panel). Tumor growth was monitored (see bottom panel, at time point day 17, from top to bottom, the curves are: untreated, CpG-TAP+Nuc-Scram, CpG-Scram+Nuc-TAP, and CpG-TAP+Nuc-TAP). Note: The dose of Nuc-TAP (third curve from top in bottom panel, at time point day 17) was reduced in order to elicit a limited antitumor effect as monotherapy in order to better show that when mice are pre-treated with CpG-TAP, but not control, siRNA, the antitumor effect is enhanced.

FIG. 10A-B shows recurrence by measuring survival. FIG. 10A is an experimental design of mice vaccinated against the induced neoantigens (CpG-TAP) and said neoantigens induced in the recurring tumors (Nucl-TAP) in a pancreatic cancer model. FIG. 10B is a survival plot showing that the combination of vaccination and induction leads to significant inhibition of recurrence/extension of survival.

FIG. 11A-C show tumor regression and survival following vaccination against future tumors with CpG-TAP siRNAs.

FIG. 11A shows an experimental system of a carcinogen-induced model for fibrosarcoma whereby mice are first treated with carcinogen, methyl cholanthrene (MCA) and tumor develop about three months later. FIG. 11B shows combination of vaccination and induction leads to a significant therapeutic impact in terms of complete tumor regression in 50% of the mice and FIG. 11C shows majority of mice surviving long-term including mice with small tumors that do not continue to grow.

FIG. 12 shows that injection of CpG-TAP siRNA to tumor bearing mice implanted with TLR9-expressing A20 B cell lymphoma tumor prevents tumor development. In the figure, at day 11, the top curve is untreated, the middle curve is CpG-Ctrl, and the bottom curve is CpG-TAP.

FIG. 13A is an experimental design showing days' post vaccination against the induce neoantigens (CpG-TAP) and said neoantigens induced in the developing tumors (Nucl-TAP). FIG. 13B shows cancer vaccination against existing, concurrent tumor and shows a decrease in tumor volume at days 4, 8, and 12 following injection of CpG-TAP siRNA when compared to untreated, Nucl-TAP, CpG-Ctrl/Nucl-TAP, CpG-TAP and CpG-TAP/Nucl-TAP.

### DETAILED DESCRIPTION

The present invention is based, in part, on the surprising discovery that targeted inhibition or downregulation of antigen processing pathways such as one or more of ERAAP, TAP, and invariant chain (Ii) specifically in cells can stimulate neoantigens and have utility in treating patients that are likely to have cancer. For instance, the present methods provide for vaccination against artificial neoantigens—which are not likely to be found in the tumors of a patient's eventual tumor—via inhibition or downregulation of one or more of ERAAP, TAP, and Ii, e.g., via a targeting, nucleic acid-based agent, to create a persistent immune response and, upon onset of a tumor, repeating inhibition or downregulation of a mediator of antigen processing, such as one or more of ERAAP, TAP, and Ii to regenerate the artificial neoantigens and direct the persistent immune response against the tumor. Accordingly, the present methods stimulate an immune response that can be tuned to a tumor as needed.

In various embodiments, the present invention provides a method of treating cancer in a subject need thereof, by administering an effective amount of an immune-modulating agent to the subject's cancer cells to direct a subject's existing immune response to a neoantigen against the cancer, where the immune-modulating agent inhibits and/or downregulates a mediator of antigen processing and induces neoantigen formation; and the subject has an existing immune response against the induced neoantigen.

In various embodiments, the present invention provides a method of treating cancer by vaccinating a subject in need with an immune modulatory agent to stimulate neoantigen-directed immune response in the patient and upon tumor development, treating the tumor with immune modulatory agent(s) to stimulate (the same) neoantigens in the tumor and direct the pre-existing immune response against the tumor.

In various embodiments, pre- and post-tumor, the immune modulatory agent(s) may be the same or different.

In various embodiments, pre- and post-tumor, the neoantigens stimulated are the same.

In various embodiments, the present invention relates to a universal vaccine having a mixture of such epitopes in the form of peptides, RNA, whole protein, DNA, etc.

In various embodiments, the present invention relates to a universal immune monitoring system for (vaccinated) patients for T cell responses against TAP, ERAAP downregulation-induced neoantigens, for example using HLA-E/neopitope tetramers.

In various embodiments, the present invention relates to a universal adoptive T cell therapy approach, one (or rather a mixture of several), such universal TCRs or CARs that will be transduced in the (any) patient's T cells and said mediators, i.e. ERAAP or TAP, downregulated in the patient's tumor by targeted delivery of corresponding siRNA.

In various embodiments, the present invention provides, without being bound by theory, an important advantage in that expression/presentation of the neoepitopes and thereby stimulation of the TCR or CAR expressing T cells is transient (e.g. because it is controlled by aptamer-targeted siRNA inhibition which is transient), and thereby reduces concerns of T cell dysfunction or toxicity.

### Methods of Cancer Treatment

In various embodiments, the present invention provides methods of altering the immune system of a subject that is susceptible to having cancer. For instance, the present methods may stimulate an immune response, e.g., a vaccine response, which can be directed against future tumors. In various embodiments, the present methods reduce the likelihood of cancer onset, progression or recurrence.

In various embodiments, the present invention provides methods of altering the immune system of a subject in cancer remission (e.g., have previously been afflicted with a cancer, e.g., having minimal residual disease (MRD)) and/or predisposed to cancer (e.g., by having one or more risk factors for cancer) as described herein. In various embodiments, the present invention provides methods of preventing onset, progression or recurrence of cancer in a susceptible subject.

In various embodiments, the present methods induce neoantigens that stimulate an immune response against a future tumor.

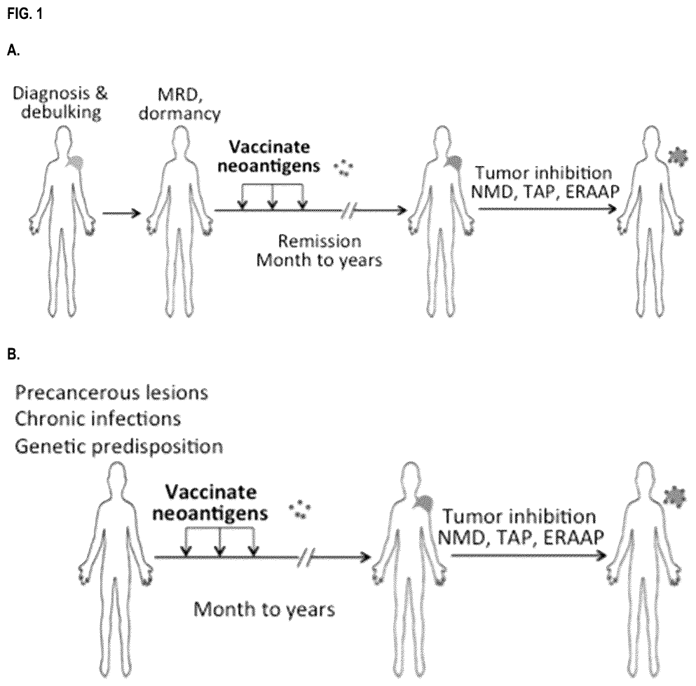

In some embodiments, the invention relates to a vaccination strategy, e.g., a transient vaccination strategy, for subjects in remission (e.g., a subject with MRD) or at risk of developing cancer that controls the growth of the future tumor. In some embodiments, the invention relates to a vaccination strategy for subjects in remission (e.g., a subject with MRD) or at risk of developing cancer against antigens that are not expressed in the patient or the individual, nor in the future tumor, and when or if tumor develops induce the same antigens in the tumor (see, e.g., FIG. 1).

In various embodiments, the present methods do not substantially trigger an autoimmune reaction or trigger only a clinically acceptable autoimmune reaction.

A schematic of illustrative embodiments is found in FIG. 1. In various embodiments, transient expression of neoantigens in a subject's tumor is stimulated. The procedure is essentially like prophylactically vaccinating against a pathogen, e.g., influenza. In this non-limiting analogy, the neoantigens (FIG. 1, dots) are the equivalent of the antigens in the flu vaccine, and the neoantigen expressing tumors the equivalent of the flu virus expressing its (neo)antigens in the infected patient. In various embodiments, the vaccination is intended to stimulate an immune response that can be directed against a tumor, should one develop. Accordingly, in various embodiments, the immune response is persistent and may require boosting. Although FIG. 1 only shows NMD, TAP, and ERAAP, it is equally applicable to Ii.

### Immune-Modulating Agents

In various embodiments, the present invention pertains to an immune-modulating agent. In various embodiments, the immune-modulating agent elicits and/or boosts an anti-tumor immune response. In various embodiments, the immune-modulating agent is a vaccine. In various embodiments, the immune-modulating agent stimulates the generation of an immune response against neoantigens. In various embodiments, the immune-modulating agent vaccinates against a neoantigen. In various embodiments, the immune-modulating agent elicits and/or boosts an anti-tumor immune response via generation of a neoantigen-mediated immune response.

In some embodiments, the immune-modulating agent induces neoantigens in tumor cells in situ.

In some embodiments, the immune-modulating agent provides tumor targeted inhibition and/or downregulation of key mediators of antigen processing pathways. In various embodiments, the immune-modulating agent provides tumor targeted inhibition and/or downregulation of ERAAP. In various embodiments, the immune-modulating agent provides tumor targeted inhibition and/or downregulation of transporter associated with antigen processing (TAP). In various embodiments, the immune-modulating agent provides tumor targeted inhibition and/or downregulation of invariant chain (Ii).

In some embodiments, the immune-modulating agent provides tumor targeted inhibition and/or downregulation of key mediators of antigen processing pathways, e.g., one or more of ERAAP, TAP, and Ii, and provides the same epitopes in the cells having the inhibition and/or downregulation (i.e. the epitope generation is not stochastic).

ERAAP is an ER-resident aminopeptidase that trims the TAP-transported peptides to optimize their association with the nascent MHC class I molecules (see Nature. 2002; 419(6906):480-3). Importantly, without wishing to be bound by theory, ERAAP deficiency induces significant alterations in the MHC class I presented peptidome. Some peptides are lost while new peptides appear, the latter probably, without wishing to be bound by theory, because they escape ERAAP processing. Like TAP-deficient cells, ERAAP-deficient cells are immunogenic in wild type mice inducing T cell response against the new ERAAP-loss induced peptides to which the wild type mouse has not been tolerized, and inhibit tumor growth. The new peptides are presented both by classical MHC class Ia molecules as well as by nonclassical MHC class Ib molecules, specifically Qa-1b. A dominant peptide presented by Qa-1b in the H-2b background was identified as FYAEATPML (FL9) derived from FAM49B protein). Qa-1b restricted presentation of the FL9 peptide stimulates CD8+ T cell responses in wild type mice that can kill ERAAP-deficient, but not ERAAP sufficient, targets.

TAP is a critical component of MHC class I presentation responsible for transporting the proteasome generated peptides from the cytoplasm to the ER where they are loaded onto the nascent MHC class I molecules (see Nat Rev Immunol. 2011; 11(12):823-36.) TAP function is frequently downregulated in tumors conceivably, without wishing to be bound by theory, to avoid immune recognition. TAP-deficient cells present novel peptide-MHC complexes resulting from alternative antigen processing pathways that are upregulated or become dominant in the absence of the canonical TAP-mediated pathway. TAP deficiency-induced peptides, referred to as “T cell epitopes associated with impaired peptide processing” or TEIPP, are presented by classical MHC class Ia molecules as well as by nonclassical Qa-1b molecules. Importantly, TAP-deficient cells or DC loaded with TEIPP peptide restricted to both the classical MHC Ia and Qa-1b can stimulate CD8+ T cell responses in wild type mice and vaccination with TEIPP loaded DC, TAP-deficient DC, or adoptive transfer of TEIPP specific CD8+ T cells was shown to inhibit the growth of TAP-deficient, but not TAP sufficient, tumors.

Invariant chain is a polypeptide involved in the formation and transport of MHC class II protein. The cell surface form of the invariant chain is known as CD74. MHC class II's path toward the cell surface involves, in the rough endoplasmic reticulum, an association between the alpha and beta chains and a Ii, which stabilizes the complex. Without the invariant chain, the alpha and beta proteins will not associate. Ii trimerizes in the ER, associates with MHC class II molecules and is released from the ER as a nine subunit complex. This MHC-invariant complex passes from the RER to, and out of, the Golgi body. Before moving to the cell surface, the vesicle containing this complex fuses with an endocytic compartment where an external protein has been broken into fragments. Here the invariant chain is proteolytically degraded and a peptide from the external protein associates with the MHC II molecule in the channel between the alpha-1 and beta-1 domains. The resulting MHC II-peptide complex proceeds to the surface where it is expressed.

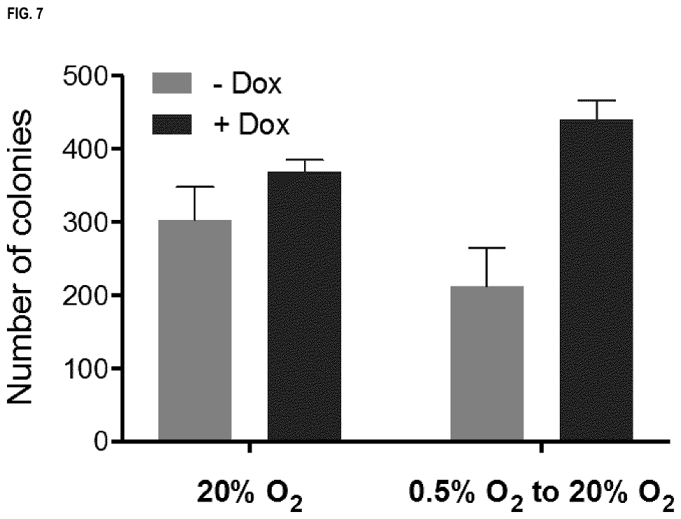

In some embodiments, the immune-modulating agent inhibits and/or downregulates a nonsense-mediated mRNA (NMD) process. NMD is an evolutionarily conserved surveillance mechanism in eukaryotic cells that prevents the expression of mRNAs containing a premature termination codon (PTC). Without wishing to be bound by theory, inhibition of results in the upregulation of several products encoded by the PTC-containing mRNAs and many of these products, resulting from aberrant splicing or NMD-dependent autoregulated alternative splicing encode new peptides that have not induced tolerance. In various embodiments, upregulation of such products when NMD is inhibited in tumor cells will elicit an immune response against (some of) the new products, and that the immune response will inhibit tumor growth. In some embodiments, the immune-modulating agent is a small interfering RNA (siRNA) which downregulates certain NMD factors (e.g., SMG1, UPF1, UPF2, UPF3, RENT1, RENT2, eIF4A, UPF1, UPF2, UPF3B, RNPS1, Y14, MAGOH, NMD1, or combinations thereof). However, as noted below, in various embodiments, inhibiting and/or downregulating one or more of ERAAP, TAP, and Ii is preferred over NMD due to the latter's possible role in enhancing tumor malignancy when inhibited in tumor cells (see FIG. 7).

In some embodiments, the immune-modulating agent comprises a small interfering RNA, or a micro RNA, or an antisense RNA.

In some embodiments, the immune-modulating agent comprises a oligonucleotide molecule, such as a small interfering RNA, or a micro RNA, or an antisense RNA which is targeted to tumor cells, e.g., by a targeting agent.

In some embodiments, the immune-modulating agent comprises a oligonucleotide molecule, such as a small interfering RNA, or a micro RNA, or an antisense RNA which is targeted to tumor cells by conjugation to an oligonucleotide aptamer ligand or a protein-based targeting agent.

In various embodiments, the immune-modulating agent produces inhibition and/or downregulation of specific mediators of an antigen processing pathway like one or more of ERAAP, TAP, and Ii and stimulates novel epitopes to which the immune system has not been tolerized and thereby they could function essentially as neoantigens. Such epitopes are non-mutated subdominant epitopes that are normally not presented and therefore carry a reduced risk of autoimmunity. Importantly, epitopes generated by downregulation of one or more of ERAAP, TAP, and Ii are not generated as a result of random events in the cell, therefore they are more like to be shared, namely the same epitope presented by any cell in which the corresponding target was downregulated.

In various embodiments, the immune-modulating agent does not substantially trigger an autoimmune reaction.

In various embodiments, the immune-modulating agent comprises a targeting agent which is specific for a desired target cell, e.g., a tumor cell (e.g., a cell of any of the cancers described herein). In various embodiments, the immune-modulating agent comprises a targeting agent such as an aptamer-oligonucleotide molecule. In some embodiments, the aptamer is specific for a desired target cell, e.g., a tumor cell (e.g., a cell of any of the cancers described herein). In various embodiments, the immune-modulating agent comprises a nucleolin aptamer. In various embodiments, the immune-modulating agent comprises an epithelial cell adhesion molecule (EpCAM) aptamer (e.g., 5′-GCGACUGGUUACCCGGUCG-3′ or variations thereof) (SEQ ID NO: 1). In various embodiments, the immune-modulating agent comprises a VEGF aptamer.

In various embodiments, the targeting agent is an antibody, antibody format, or paratope-comprising fragment thereof directed against the analyte of interest. In various embodiments, the antibody is a full-length multimeric protein that includes two heavy chains and two light chains. Each heavy chain includes one variable region (e.g., V_{H}) and at least three constant regions (e.g., CH₁, CH₂ and CH₃), and each light chain includes one variable region (V_{L}) and one constant region (C_{L}). The variable regions determine the specificity of the antibody. Each variable region comprises three hypervariable regions also known as complementarity determining regions (CDRs) flanked by four relatively conserved framework regions (FRs). The three CDRs, referred to as CDR1, CDR2, and CDR3, contribute to the antibody binding specificity. In some embodiments, the antibody is a chimeric antibody. In some embodiments, the antibody is a humanized antibody.

In some embodiments, the targeting agent is an antibody derivative or format. In some embodiments, the targeting agent comprises a targeting moiety which is a single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; a peptide aptamer; an alterases; a plastic antibodies; a phylomer; a stradobody; a maxibody; an evibody; afynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, a F(ab′)₂, a peptide mimetic molecule, or a synthetic molecule, as described in U.S. Pat. Nos. or Patent Publication Nos. U.S. Pat. No. 7,417,130, US 2004/132094, U.S. Pat. No. 5,831,012, US 2004/023334, U.S. Pat. Nos. 7,250,297, 6,818,418, US 2004/209243, U.S. Pat. Nos. 7,838,629, 7,186,524, 6,004,746, 5,475,096, US 2004/146938, US 2004/157209, U.S. Pat. Nos. 6,994,982, 6,794,144, US 2010/239633, U.S. Pat. No. 7,803,907, US 2010/119446, and/or U.S. Pat. No. 7,166,697, the contents of which are hereby incorporated by reference in their entireties. See also, Storz MAbs. 2011 May-June; 3(3): 310-317.

In some embodiments, the targeting agent is a peptide directed to a cell or marker of interest.

In various embodiments, the oligonucleotide molecule comprises at least one of a short interfering RNA (siRNA); a micro-interfering RNA (miRNA); antisense oligonucleotides; a small, temporal RNA (stRNA); a short, hairpin RNA (shRNA), and antisense RNA, or combinations thereof. In various embodiments, the oligonucleotide molecule targets specific mediators of an antigen processing pathway like one or more of ERAAP, TAP, and Ii.

In various embodiments, the immune-modulating agent comprises a molecule suitable for RNA interference, i.e. the process of sequence-specific post-transcriptional gene silencing in animals mediated by short interfering RNAs (siRNAs). In various embodiments, the immune-modulating agent comprises a siRNA.

The presence of long dsRNAs in cells stimulates the activity of a ribonuclease Ill enzyme referred to as dicer. Dicer is involved in the processing of the dsRNA into short pieces of dsRNA known that are siRNAs. siRNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNAs duplex. Accordingly, some embodiments of the invention contemplate use of dsRNA to downregulate protein expression from mRNA.

In various embodiments, the present siRNA are between about 18-30 basepairs (e.g., about 18, or about 19, or about 20, or about 21, or about 22, or about 23, or about 24, or about 25, or about 26, or about 27, or about 28, or about 29, or about 30 basepairs) and induce the RNA interference (RNAi) pathway. In some embodiments, the siRNAs are 21mers with a central 19 bp duplex region and symmetric 2-base 3-overhangs on the termini, although other variations of length and overhang are possible.

The strands of a double-stranded interfering RNA (e.g., a siRNA) may be connected to form a hairpin or stem-loop structure (e.g., a shRNA). Thus, the dsRNA of some embodiments of the invention may also be a hairpin or short hairpin RNA (shRNA).

In various embodiments, the immune-modulating agent comprises a miRNA. MiRNAs are short nucleic acid molecules that are able to regulate the expression of target genes. See review by Carrington et al. Science, Vol. 301(5631):336-338, 2003. MiRNAs are often between about 18 to about 24 nucleotides in length. MiRNAs act as repressors of target mRNAs by promoting their degradation, when their sequences are perfectly complementary, and/or by inhibiting translation, when their sequences contain mismatches. Without being bound by theory, mature miRNAs are believed to be generated by pol II or pol Ill and arise from initial transcripts termed -miRNAs. These pri-miRNAs are frequently several thousand bases long and are therefore processed to make much shorter mature miRNAs. These pri-miRNAs may be multicistronic and result from the transcription of several clustered sequences that organize what may develop into many miRNAs. The processing to yield miRNAs may be two-steps. First, pri-miRNAs may be processed in the nucleus by the RNase Drosha into about 70- to about 100-nucleotide hairpin-shaped precursors (pre-miRNAs). Second, after transposition to the cytoplasm, the hairpin pre-miRNAs may be further processed by the RNase Dicer to produce a double-stranded miRNA. The mature miRNA strand may then be incorporated into the RNA-induced silencing complex (RISC), where it may associate with its target mRNAs by base-pair complementarity and lead to suppression of protein expression. The other strand of the miRNA duplex that is not preferentially selected for entry into a RISC silencing complex is known as the passenger strand or minor miRNA or star (*) strand. This strand may be degraded. It is understood that, unless specified, as used herein an miRNA may refer to pri- and/or pre- and/or mature and/or minor (star) strand and/or duplex version of miRNA.

In various embodiments, the immune-modulating agent comprises an antisense oligonucleotide. An antisense oligonucleotide is a nucleic acid strand (or nucleic acid analog) that is complementary to an mRNA sequence. Antisense occurs naturally and can trigger RNA degradation by the action of the enzyme RNase H. In various embodiments, the antisense oligonucleotide is non-naturally occurring. In various embodiments, the antisense oligonucleotide comprises one or more nucleic acid analogs. In various embodiments, the antisense oligonucleotide is nuclease resistant and activates RNase H. In various embodiments, the antisense oligonucleotide comprises phosphorothioate RNA and other nucleic acid analogs that bind to RNA and sterically inhibit processes without activating RNase H (such as 2′-O-methyl phosphorothioate RNA, Morpholino oligos, locked nucleic acids, or peptide nucleic acids). These latter RNase-H independent oligos do not trigger degradation of mRNA but they can be to block translation, alter splicing of pre-mRNA, inhibit activity of miRNA, block ribozyme activity, and interfere with various other processes that require some other factor to bind to a particular sequence on an RNA molecule.

In various embodiments, the immune-modulating agent is one of US Patent Publication No. 2012/0263740, the entire contents of which are hereby incorporated by reference.

In some embodiments, the oligonucleotide molecule and/or targeting agent, such as a aptamer, has one or more nucleotide substitutions (e.g., at least one of adenine, guanine, thymine, cytosine, uracil, purine, xanthine, diaminopurine, 8-oxo-N⁶-methyladenine, 7-deazaxanthine, 7-deazaguanine, N⁴,N⁴-ethanocytosin, N⁶,N⁶-ethano-2,6-diaminopurine, 5-methylcytosine, 5-(C³-C⁶)-alkynylcytosine, 5-fluorouracil, 5-bromouracil, pseudoisocytosine, 2-hydroxy-5-methyl-4-triazolopyridin, isocytosine, isoguanin, inosine, non-naturally occurring nucleobases, locked nucleic acids (LNA), peptide nucleic acids (PNA), variants, mutants, analogs or combinations thereof.

In various embodiments, the aptamer and/or the siRNA (e.g., the sense strand) comprise fluoro-modified pyrimidines, e.g., 2′-fluoro-modified pyrimidines, e.g., one or more of 2′-fluoro-cytosine (C), 2′-fluoro-thymine (T), and 2′-fluorouracil (U).

In some embodiments, any immune-modulating agent (and/or additional agents) described herein is formulated in accordance with procedures as a composition adapted for a mode of administration described herein.

In some embodiments, the present invention provides vaccination with lysate loaded ex vivo generated dendritic cells. In some embodiments, the lysate is generated from the subject's normal tissue in which one or more of ERAAP, TAP, and Ii is downregulated, e.g., by nucleolin-siRNA or by shRNA expressing lentiviral vectors. Sources of normal tissue can be fibroblasts or B cells that can be readily expanded in vitro in short term cultures. Instead of lysate, RNA from the tumor, total or mRNA enriched poly A+RNA may be used. Poly A+RNA can be also amplified to generate sufficient antigen for DC loading and thereby limit the ex vivo culture step.

In some embodiments, the present invention provides vaccination with neoantigen mRNA-lipid nanocarriers. In some embodiments, vaccination with mRNA complexed to lipid carriers like DOPE and DOTMA can be undertaken (Nature. 2016; 534(7607):396-401). Illustrative lipid carriers include 1,2-Dioleoyl-sn-glycero-3-phosphatidylcholine (DOPC), 1,2-Dioleoyl-sn-glycero-3-phosphatidylethanolamine (DOPE), cholesterol, N-[1-(2,3-Dioleyloxy)propyl]N,N,N-trimethylammonium chloride (DOTMA), 1,2-Dioleoyloxy-3-trimethylammonium-propane (DOTAP), Dioctadecylamidoglycylspermine (DOGS), N-(3-Aminopropyl)-N,N-dimethyl-2,3-bis(dodecyloxy)-1-propanaminium bromide (GAP-DLRIE), cetyltrimethylammonium bromide (CTAB), 6-lauroxyhexyl ornithinate (LHON), 1-)2,3-Dioleoloxypropyl)2,4,6-trimethylpyridinium (20c), 2,3-Dioleyloxy-N-[2(sperminecarboxamido)-ehtyl]-N,N-dimethyl-1-propanaminium trifluoroacetate (DOSPA), 1,2-Dioleyl-3-trimethylammonium-propane (DOPA), N-(2-Hydroxyethyl)-N,N-dimethyl-2,3-bis(tetradecyloxy)-1-propanaminium bromide (MDRIE), Dimyristooxypropyl dimethyl hydroxyethyl ammonium bromide (DMRI), 3β-[N—(N′,N′-Dimethylaminoethane)-carbamoyl]cholesterol (DC-Chol), Bis-guanidium-tren-cholesterol (BGTC), 1,3-Dioleoxy-2-(6-carboxy-spermyl)-propylamide (DOSPER), Dimethyloctadecylammonium bromide (DDAB), Dioctadecylamidoglicylspermidin (DSL), rac-[(2,3-Dioctadecyloxypropyl)(2-hydroxyethyl)]-dimethylammonium chloride (CLIP-1), rac-[2(2,3-Dihexadecyloxypropyl-oxymethyloxy)ehtyl]trimethylammonium chloride (CLIP-6), Ethyldimyrisotylphosphatidylcholine (EDMPC), 1,2-Distearyloxy-N,N-dimethyl-3-aminopropane (DSDMA), 1,2-Dimyristoyl-trimethylammoniumpropane (DMTAP), O,O′-Dimyristyl-N-lysyl asparate (DMKE), 1,2-Distearoyl-sn-glycero-3-ethylphosphocholine (DSEPC), N-Palmitoyl-D-erythro-spingosyl carbamoyl-spermine (CCS), N-t-Butyl-No-tetradecyl-3-tetradecylaminopropionamidine (diC14-amidine), Octadecenolyoxy[ethyl-2-heptadecenyl-3 hydroxyethyl]imidazolinium chloride (DOTIM), N1-Cholesteryloxycarbonyl-3,7-diazanonane-1,9-diamine (CDAN) and 2-(3-[Bis-(3-amino-propyl)-amino]propylamino)-N-ditetradecylcarbamoylme-ethyl-acetamide (RPR2091290). In some embodiments, this approach will be used to vaccinate against neoantigens using total RNA, mRNA enriched poly A+ RNA, or amplified polyA+ RNA from syngeneic fibroblasts or B cells as described above.

In some embodiments, the present invention provides inducing neoantigens in DC in situ (optionally via one or more of CpG, DEC205, and CD40). In some embodiments, expression of the neoantigens in the DC in situ is undertaken. In some embodiments, the neoantigen inducing siRNA (to inhibit one or more of ERAAP, TAP, and Ii) is targeted to DC by conjugating the siRNAs to a DEC205 aptamer or a TLR9 stimulating CpG oligonucleotide (ODN). DEC205 is a lectin-like receptor expressed on immature DC that is responsible for the uptake and cross-presentation of apoptotic cells to both CD4+ and CD8+ T cells. DEC205 conjugated antigens stimulate potent T cell responses in mice, provided a DC maturation agent is included in the protocol like CD40 antibody, poly I:C or CpG. A DEC205 aptamer that was shown to target the OVA antigen to DC in vitro and in vivo will be used. DEC205-siRNA conjugates will be characterized in vitro for DEC205 dependent downregulation of their corresponding targets in DC and the consequences on their functionality, namely improved stimulation of antigen-specific T cell responses. Validated DEC205 aptamer-siRNA conjugates may be used in mouse immunotherapy experiments by administration into the circulation via tail vein injection together with the well characterized 1680 phosphorothioate CpG ODN. Conditions in terms of regimen, dose, or alternative adjuvants like poly I:C, may be evaluated using DEC205-ERAAP siRNA and measuring the induction of CD8+ T cell responses against a defined ERAAP deficient-induced epitope, the Qa-1b restricted FYAEATPML (FL9) peptide derived from FAM49B protein. Alternatively, the siRNA will be targeted by conjugation to a CpG ODN. CpG ODNs have been successfully used to target STAT3 siRNA to DC in situ. Other embodiments provide for co-delivery of unconjugated siRNAs and CpG ODN or Poly I:C as DC maturation agents to DC in situ by encapsulation in the anionic lipoplexes discussed herein. Further, DC targeting may be mediated by directing to CD40 using the targeting methods described herein (including, without limitation, antibody- and aptamer-based approached). DC targeting by CD40, in some embodiments, directs to CD40 upregulated on DCs and can activate DCs.

Routes of administration include, for example: intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. In some embodiments, the administering is effected orally or by parenteral injection.

Any immune-modulating agent (and/or additional agents) described herein can be administered parenterally. Such immune-modulating agents (and/or additional agents) can also be administered by any other convenient route, for example, by intravenous infusion or bolus injection, by absorption through epithelial or mucocutaneous linings (e.g., oral mucosa, rectal and intestinal mucosa, etc.) and can be administered together with another biologically active agent. Administration can be systemic or local. Various delivery systems are known, e.g., encapsulation in liposomes, microparticles, microcapsules, capsules, etc., and can be used to administer.

Dosage forms suitable for parenteral administration (e.g., intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g., lyophilized composition), which can be dissolved or suspended in sterile injectable medium immediately before use. They may contain, for example, suspending or dispersing agents known in the art.

### Subjects

In many embodiments, the subject of the present invention is at risk for presenting with cancer in the future. In many embodiments, the subject of the present invention is likely to be afflicted by a cancer. In many embodiments, the subject of the present invention is characterized by one or more of a high risk for a cancer, a genetic predisposition to a cancer (e.g., genetic risk factors), a previous episode of a cancer (e.g., new cancers and/or recurrence), a family history of a cancer, and exposure to a cancer-inducing agent (e.g., an environmental agent or an infectious agent).

In some embodiments, a subject is likely to be afflicted by cancer if the subject is characterized by a high risk for a cancer.

In some embodiments, a subject is likely to be afflicted by cancer if the subject is characterized by a genetic predisposition to a cancer. In some embodiments, a genetic predisposition to a cancer is a genetic risk factor, as is known in the art. Such risk factors may include, by way of example, HNPCC, MLH1, MSH2, MSH6, PMS1, PMS2 for at least colon, uterine, small bowel, stomach, urinary tract cancers. In some embodiments, a subject is likely to be afflicted by cancer if the subject is characterized by a previous episode of a cancer.

With developments in genomic research, correlations are being established between genetic profiles and risk for developing any number of diseases, including specific cancers. Accordingly, a powerful tool for selecting suitable subjects can be based on known or future laboratory or clinical techniques established for assessing existing genetic indicators or for monitoring changes in such indicators.

For example, a subject may have a mutation, e.g., a loss or reduction in function, in a tumor suppressor gene, or antioncogene. In some embodiments, the subject has a first “hit”, e.g., in a tumor suppressor gene, with reference to the Knudson “two-hit hypothesis.” For example, a subject may have a mutation or overexpression of an oncogene.

In one example, inherited alterations in the genes called BRCA1 and BRCA2 are involved in many cases of hereditary breast and ovarian cancer. Women with an altered BRCA1 or BRCA2 gene are 3 to 7 times more likely to develop breast cancer than women without alterations in those genes. Men with an altered BRCA1 or BRCA2 gene also have an increased risk of breast cancer (primarily if the alteration is in BRCA2), and possibly prostate cancer. Alterations in the BRCA2 gene have also been associated with an increased risk of lymphoma, melanoma, and cancers of the pancreas, gallbladder, bile duct, and stomach in some men and women. Accordingly, in some embodiments, the subject has alterations in one or more of BRCA1 and BRCA2.

For example, in the context of breast cancer, any one of the following risk factors may be useful in selecting a subject for cancer prevention with the agents described herein: gender (e.g., breast cancer is more common in females over males); aging (e.g., breast cancer is more prevalent with increased age); genetic risk factors (by way of limiting example, the presence of an alteration (e.g., mutation) in one or more of BRCA1 and BRCA2, ATM (e.g., inheriting a single mutated copy of this gene), HER2 (e.g., for breast or ovarian cancer), TP53 (e.g., subjects afflicted by Li-Fraumeni syndrome), CHEK2 (e.g., subjects afflicted by Li-Fraumeni syndrome), PTEN (e.g., subjects afflicted by Cowden syndrome), CDH1, STK11 (e.g., subjects afflicted by Peutz-Jeghers syndrome); family history of breast cancer (e.g., having one first-degree relative (e.g., mother, sister, or daughter) with breast cancer approximately doubles a woman's risk); personal history of breast cancer; race and ethnicity; features of the breast tissues (e.g., the presence of dense breast tissue, such as those caused by, for example, age, menopausal status, the use of drugs (such as menopausal hormone therapy), pregnancy, and genetics); various benign breast conditions (e.g., non-proliferative lesions (including but not limited to fibrosis and/or simple cysts (e.g., fibrocystic disease or changes), mild hyperplasia, adenosis (e.g., non-sclerosing), ductal ectasia, phyllodes tumor (e.g., benign), one or more papilloma, fat necrosis, periductal fibrosis, squamous and apocrine metaplasia, epithelial-related calcifications, mastitis, other benign tumors (including but not limited to lipoma, hamartoma, hemangioma, neurofibroma, adenomyoepthelioma), proliferative lesions without atypia (e.g., usual ductal hyperplasia, fibroadenoma, sclerosing adenosis, several papillomas (called papillomatosis), and radial scar), proliferative lesions with atypia (e.g., atypical ductal hyperplasia (ADH) and atypical lobular hyperplasia (ALH))); presence of lobular carcinoma in situ (LCIS) increased numbers of menstrual periods, previous chest radiation, carcinogen exposure (e.g., diethylstilbestrol exposure).

In some embodiments, the present subject has one or more alterations (e.g., mutations) in genes are also associated with hereditary breast and/or ovarian cancer including PALB2, CHEK2, ATM, BRIP1, RAD51C, and RAD51D.

In some embodiments, the present subject has Lynch syndrome, a hereditary cancer syndrome that increases risks of many cancers, including ovarian cancer.

In some embodiments, the present subject expresses Muc-1 on precancerous and cancerous lesions of multiple cancers including breast and colon cancer, or one or more mammary tissue-specific antigens like a-lactalbumin, Her-2, IGFBP2 and IGFIR.

In various embodiments, the subject has one or more alterations (e.g., mutations) in one or more of TP53, PIK3CA, PTEN, RBI, KRAS, NRAS, BRAF, CDKN2A, FBXW7, ARID1A, MLL2, STAG2, ATM, CASP8, CTCF, ERBB3, HLA-A, HRAS, IDH1, NF1, NFE2L2, and PIK3R1.

In various embodiments, the subject has one or more alterations (e.g., mutations) in PTCH, e.g., increasing risk of medulloblastoma, or NF1, e.g., increasing risk of neurofibroma.

In various embodiments, the subject has one or more alterations (e.g., mutations) in p27Kip1, a cell-cycle inhibitor, in which mutation of a single allele causes increased carcinogen susceptibility.

In some embodiments, the present methods are particularly useful in subjects that are in cancer remission (e.g., have previously been afflicted with a cancer, e.g., having minimal residual disease (MRD)) In some embodiments, the subject has been afflicted with 1, or 2, or 3, or 4, or 5, or 6, previous episodes of cancer. In some embodiments, the subject is at risk for a cancer recurrence.

In some embodiments, the present methods are particularly useful in subjects that have a premalignant lesion.

In some embodiments, the present methods are particularly useful in subjects that have dysplasia or “precancer” or carcinoma in situ. In some embodiments, the present methods are particularly useful in subjects that have one or more of actinic keratosis, Barrett's esophagus, atrophic gastritis, ductal carcinoma in situ, dyskeratosis congenita, sideropenic dysphagia, lichen planus, oral submucous fibrosis, solar elastosis, cervical dysplasia, leukoplakia, erythroplakia, and the like.

In some embodiments, a subject is likely to be afflicted by cancer if the subject is characterized by a family history of a cancer. In some embodiments, a parent and/or grandparent and/or sibling and/or aunt/uncle and/or great aunt/great uncle, and/or cousin has been or is afflicted with a cancer. For instance, such embodiments are particularly applicable to cancers that are often linked to family history, such as prostate, breast, colorectal, lung, ovarian and endometrial cancers. In some embodiments, a subject is likely to be afflicted by hereditary breast and ovarian cancer (HBOC).

In some embodiments, a subject is likely to be afflicted by cancer as the subject is characterized by exposure to a cancer-inducing agent (e.g., an environmental agent). For example, exposing skin to strong sunlight is a risk factor for skin cancer. By way of example, smoking is a risk factor for cancers of the lung, mouth, larynx, bladder, kidney, and several other organs.

In specific embodiments, the present invention provides prevention of a cancer induced by a carcinogen. For instance, suitable subjects include those who smoke and/or who are or have been exposed, e.g., occupationally, to asbestos or other compounds known to potentially cause cancers, for instance cancer of the lung. Chimneysweepers or factory workers handling dusts such as in the cement industry, in facilities that use fine silica or carbon particles, organic or polymeric materials, and others people routinely exposed to materials that are known or are suspected for causing cancers also can be selected. Another category suitable for vaccination are the people with significant sun exposure due to their occupation, e.g., farmers and construction workers in sub-tropical and tropical climates, as well as subjects with congenital and other nevi and other skin lesions, known to have higher incidence of malignant transformation.

In some embodiments, the subject is exposed to a carcinogen. In some embodiments, the present invention includes selecting a subject that has been or will be exposed to a carcinogen.

In some embodiments, the carcinogen is one that is classified by the International Agency for Research on Cancer's (IARC) Monographs on the Evaluation of Carcinogenic Risks to Humans, including, Group 1 carcinogens (agents that are definitely carcinogenic to humans. The exposure circumstance entails exposures that are carcinogenic to humans), Group 2A carcinogens (agents that are probably carcinogenic to humans. The exposure circumstance entails exposures that are probably carcinogenic to humans), Group 2B carcinogens (agents that are possibly carcinogenic to humans. The exposure circumstance entails exposures that are possibly carcinogenic to humans), Group 3 carcinogens (agents that are not classifiable as to its carcinogenicity to humans) and Group 4 carcinogens (agents that are probably not carcinogenic to humans). Non-limiting illustrative carcinogens include dioxins and dioxin-like compounds, benzene, kepone, EDB, asbestos, industrial smoke and tobacco smoke, benzo[a]pyrene, nitrosamines (such as nitrosonornicotine), and reactive aldehydes (such as formaldehyde), vinyl chloride, arsenic, asbestos, cadmium, hexavalent chromium(VI) compounds, Diesel exhaust, Ethylene oxide, Nickel, Radon and its decay products, Radium-226, Radium-224, Plutonium-238, Plutonium-239, and other alpha particle emitters with high atomic weight, etc.

In some embodiments, the carcinogen is an “absolute carcinogen,” i.e. it may not only initiate tumor formation but also to promote its progression.

In various embodiments, the subject is afflicted with a chronic infection. Subjects infected with hepatitis B, hepatitis C, and human papilloma viruses as well as subjects infected with H. pylori bacteria are at higher risk of developing cancers and could be another category of subjects suitable for vaccination.

A depressed immune system, such as can be found in HIV-positive or AIDS subjects, transplant recipients, geriatric subjects and so forth, can be another criterion for selecting suitable subjects.

The term subject, as used herein unless otherwise defined, is a mammal, e.g., a human. Experimental animals are also included, such as a mouse, rat, guinea pig, dog, cat, horse, cow, pig, or non-human primate, such as a monkey, chimpanzee, or baboon. In one embodiment, the subject is a veterinary patient, including the animals described herein. In one embodiment, the subject is a human.

The method also can be practiced in entirely healthy subjects who are not known to be at risk.

Another aspect of this invention applies to immunotherapy of subjects who already have cancer, e.g., a cancer that manifests through solid tumors, such as described above. One example would be a subject who has achieved remission from his/her cancer through surgery, chemotherapy, and/or radiation, or by other means. This aspect of the invention provides for prevention of cancer recurrence in such a subject.

In some embodiments, the present invention relates to a method for treating, ameliorating, or preventing cancer growth, survival, metastasis, epithelial-mesenchymal transition, immunologic escape or recurrence, comprising administering by administering an immune-modulating agent described herein. Also provided herein is a method of reducing cancer recurrence, comprising administering to a subject in need thereof an immune-modulating agent described herein. The method may also prevent cancer recurrence. The cancer may be an oncological disease. The cancer may be a dormant tumor, which may result from the metastasis of a cancer. The dormant tumor may also be left over from surgical removal of a tumor. The cancer recurrence may for example, be tumor regrowth, a lung metastasis, or a liver metastasis.

In various embodiments, the cancer is one or more of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

Another aspect of this invention applies to immunotherapy of subjects who already have cancer, e.g., a cancer that manifests through solid tumors, such as described above. In some embodiments, the subject is not in remission. In some embodiments, subjects with existing measurable disease, such as subjects that cannot be induced into remission, and/or in the neoadjuvant setting before debulking are therapeutically vaccinated.

In various embodiments, there is provided co-administration of the present immune modulating agent with one or more additional therapeutic agents. Such co-administration does not require the therapeutic agents to be administered to the subject by the same route of administration. Rather, each therapeutic agent can be administered by any appropriate route, for example, parenterally or non-parenterally. Further, co-administration relates to simultaneous or sequential administration.

In some embodiments, the immune modulating agent described herein acts synergistically when co-administered with an additional therapeutic agent. In such embodiments, the immune modulating agent and the additional therapeutic agent may be administered at doses that are lower than the doses employed when the agents are used in the context of monotherapy.

Further, in various embodiments, the present methods relate to treating a subject who has previously undergone treatment with an additional therapeutic agent. Further, in various embodiments, the present methods relate to treating a subject who is presently undergoing treatment with an additional therapeutic agent.

In some embodiments, the present invention pertains to chemotherapeutic agents as additional therapeutic agents. For example, without limitation, such combination of the present immune modulating agents and chemotherapeutic agent find use in the treatment of cancers, as described elsewhere herein. Examples of chemotherapeutic agents include, but are not limited to, alkylating agents such as thiotepa and CYTOXAN cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (e.g., bullatacin and bullatacinone); a camptothecin (including the synthetic analogue topotecan); bryostatin; cally statin; CC-1065 (including its adozelesin, carzelesin and bizelesin synthetic analogues); cryptophycins (e.g., cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegaII (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN doxorubicin (including morpholino- doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as minoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL paclitaxel (Bristol-Myers Squibb Oncology, Princeton, N.J.), ABRAXANE Cremophor-free, albumin-engineered nanoparticle formulation of paclitaxel (American Pharmaceutical Partners, Schaumberg, 111.), and TAXOTERE doxetaxel (Rhone-Poulenc Rorer, Antony, France); chloranbucil; GEMZAR gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE. vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb); inhibitors of PKC-α, Raf, H-Ras, EGFR (e.g., erlotinib (Tarceva)) and VEGF-A that reduce cell proliferation and pharmaceutically acceptable salts, acids or derivatives of any of the above. In addition, the methods of treatment can further include the use of radiation. In addition, the methods of treatment can further include the use of photodynamic therapy.

In some embodiments, the present invention relates to combination therapy with one or more agents that modulate immune checkpoint. In various embodiments, the immune checkpoint agent targets one or more of PD-1, PD-L1, and PD-L2. In various embodiments, the immune-modulating agent is PD-1 inhibitor. In various embodiments, the immune-modulating agent is an antibody specific for one or more of PD-1, PD-L1, and PD-L2. For instance, in some embodiments, the immune-modulating agent is an antibody such as, by way of non-limitation, nivolumab, (ONO-4538/BMS-936558, MDX1106, OPDIVO, BRISTOL MYERS SQUIBB), pembrolizumab (KEYTRUDA, MERCK), pidilizumab (CT-011, CURE TECH), MK-3475 (MERCK), BMS 936559 (BRISTOL MYERS SQUIBB), MPDL3280A (ROCHE). In some embodiments, the immune-modulating agent is an agent that targets one or more of CTLA-4, AP2M1, CD80, CD86, SHP-2, and PPP2R5A. In various embodiments, the immune-modulating agent is an antibody specific for one or more of CTLA-4, AP2M1, CD80, CD86, SHP-2, and PPP2R5A. For instance, in some embodiments, the immune-modulating agent is an antibody such as, by way of non-limitation, ipilimumab (MDX-010, MDX-101, Yervoy, BMS) and/or tremelimumab (Pfizer). In some embodiments, the immune-modulating agent targets one or more of CD137 (4-1BB) or CD137L. In various embodiments, the immune-modulating agent is an antibody specific for one or more of CD137 (4-1BB) or CD137L. For instance, in some embodiments, the immune-modulating agent is an antibody such as, by way of non-limitation, urelumab (also known as BMS-663513 and anti-4-1BB antibody).

In some embodiments, the present immune modulating agents potentiate treatment with one or more the immune checkpoint agents.

### Definitions

As used herein, “a,” “an,” or “the” can mean one or more than one.

Further, the term “about” when used in connection with a referenced numeric indication means the referenced numeric indication plus or minus up to 10% of that referenced numeric indication. For example, the language “about 50” covers the range of 45 to 55.

An “effective amount,” when used in connection with medical uses is an amount that is effective for providing a measurable treatment, prevention, or reduction in the rate of pathogenesis of a disease of interest.

As used herein, something is “decreased” if a read-out of activity and/or effect is reduced by a significant amount, such as by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100%, in the presence of an agent or stimulus relative to the absence of such modulation. As will be understood by one of ordinary skill in the art, in some embodiments, activity is decreased and some downstream read-outs will decrease but others can increase.

Conversely, activity is “increased” if a read-out of activity and/or effect is increased by a significant amount, for example by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100% or more, at least about 2-fold, at least about 3-fold, at least about 4-fold, at least about 5-fold, at least about 6-fold, at least about 7-fold, at least about 8-fold, at least about 9-fold, at least about 10-fold, at least about 50-fold, at least about 100-fold, in the presence of an agent or stimulus, relative to the absence of such agent or stimulus.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.” As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

The amount of compositions described herein needed for achieving a therapeutic effect may be determined empirically in accordance with conventional procedures for the particular purpose. Generally, for administering therapeutic agents for therapeutic purposes, the therapeutic agents are given at a pharmacologically effective dose. A “pharmacologically effective amount,” “pharmacologically effective dose,” “therapeutically effective amount,” or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. Therapeutic benefit also includes halting or slowing the progression of the underlying disease or disorder, regardless of whether improvement is realized.

Effective amounts, toxicity, and therapeutic efficacy can be determined by standard pharmaceutical procedures in cell cultures or experimental animals, e.g., for determining the LD50 (the dose lethal to about 50% of the population) and the ED50 (the dose therapeutically effective in about 50% of the population). The dosage can vary depending upon the dosage form employed and the route of administration utilized. The dose ratio between toxic and therapeutic effects is the therapeutic index and can be expressed as the ratio LD50/ED50. In some embodiments, compositions and methods that exhibit large therapeutic indices are preferred. A therapeutically effective dose can be estimated initially from in vitro assays, including, for example, cell culture assays. Also, a dose can be formulated in animal models to achieve a circulating plasma concentration range that includes the IC50 as determined in cell culture, or in an appropriate animal model. Levels of the described compositions in plasma can be measured, for example, by high performance liquid chromatography. The effects of any particular dosage can be monitored by a suitable bioassay. The dosage can be determined by a physician and adjusted, as necessary, to suit observed effects of the treatment.

In certain embodiments, the effect will result in a quantifiable change of at least about 10%, at least about 20%, at least about 30%, at least about 50%, at least about 70%, or at least about 90%. In some embodiments, the effect will result in a quantifiable change of about 10%, about 20%, about 30%, about 50%, about 70%, or even about 90% or more. Therapeutic benefit also includes halting or slowing the progression of the underlying disease or disorder, regardless of whether improvement is realized.

As used herein, “methods of treatment” are equally applicable to use of a composition for treating the diseases or disorders described herein and/or compositions for use and/or uses in the manufacture of a medicaments for treating the diseases or disorders described herein.

This invention is further illustrated by the following non-limiting examples.

### EXAMPLES

### Example 1: Induction of Neoantigens by Tumor Targeted Inhibition of ERAAP- and TAP-Tumor Targeting Via Nucleolin

Given the paucity of known receptors expressed on human tumor cells that can be used for in vivo targeting, identification of endogenous targets that are broadly expressed on tumor cells of distinct origin was undertaken. Nucleolin, normally present in the nucleolar compartment as well as the cytoplasm, is translocated to the cell surface on many if not all tumor cells of both murine and human origin. A nucleolin binding aptamer was generated (see Exp Mol Pathol. 2009; 86(3):151-64) that recognizes both murine and human nucleolin, which at high doses can be cytotoxic.

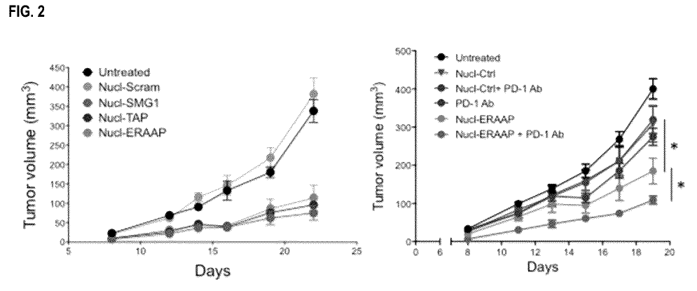

We determined that the nucleolin aptamer, used at ˜100 fold reduced dose compared to what is needed to directly inhibit tumor cells, can efficiently target the NMD specific Smg-1 siRNA to tumor cells of distinct origin, 4T1 breast carcinoma, CT26 colon carcinoma, A20 B-lymphoma, as well as to tamoxifen-induced BRAF-resistant melanoma. Nucleolin aptamer-ERAAP and TAP siRNA conjugates were generated and characterized. Intravenous injection of the nucleolin-targeted ERAAP or TAP conjugates inhibited the growth of subcutaneously implanted palpable 4T1 tumors that was comparable to that of NMD inhibition (FIG. 2 left panel, Nucl-Smg1). As shown in FIG. 2 right panel, ERAAP inhibition potentiated the inhibitory effect of PD-1 Ab. These experiments, therefore provide evidence that tumor targeted ERAAP or TAP inhibition represent an alternative approach to induce neoantigens in tumor cells in situ. Of note, 4T1 breast carcinoma is an aggressive, poorly immunogenic tumor that is notoriously difficult to treat. Few if any immune based monotherapies can impact palpable subcutaneously implanted 4T1 tumors. Thus the antitumor effects seen in FIG. 2 are indicative of the potency of neoantigen induction strategies tested in these studies.

### Example 2: Tumor-Targeted NMD and ERAAP Downregulation Inhibits Tumor Growth in the BRAF/PTEN Melanoma Tumor Model

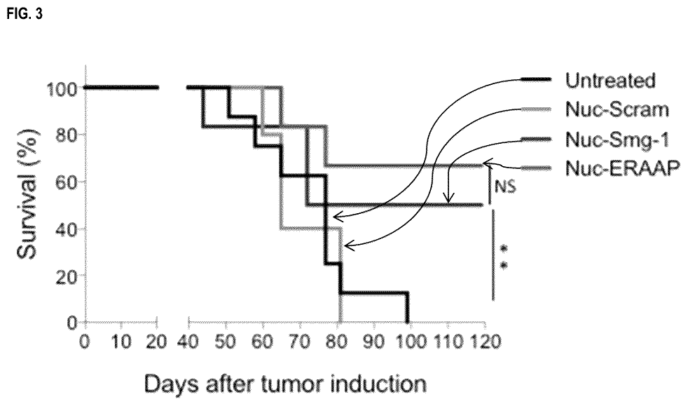

To enhance the relevance and predictive value of preclinical models for cancer immunotherapy the tamoxifen-induced BRAF mutant BRAF/PTEN model (Nature Genetics. 2009; 41(5):544-52) was used. Neoantigens were induced in the tumor bearing mice about 3 weeks post tamoxifen application (palpable tumors 2-3 mm height) by nucleolin targeted inhibition of NMD (Smg-1 siRNA) or ERAAP. As shown in FIG. 3, over 50% of mice survived over 110 days in the NMD or ERAAP treated groups, rather unprecedented in this challenging model. In two mice tumors regressed (NMD inhibition, n=2; ERAAP inhibition, n=1). Further altering the aptamer conjugates, treatment conditions, and increasing intensity of treatment in terms of dose and number of treatments, is likely to significantly enhance the antitumor impact shown in this experiment.

### Example 3: Prophylactic Vaccination Against Cancer

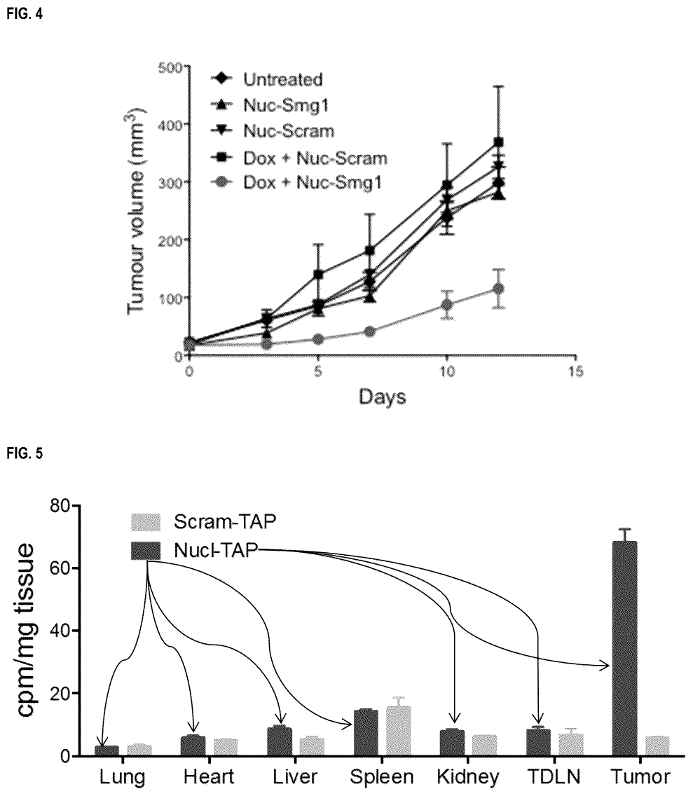

Above, a new paradigm for prophylactic cancer vaccination, “prorapeutic” vaccination (see, e.g., FIG. 1, panel B) is described (it is noted that this paradigm also applies to recurring tumors). To test the idea mice were vaccinated with irradiated CT26 colorectal tumor cells expressing the NMD factor Smg-1 shRNA under doxycyline DOX control which induces neoantigens and confers protective antitumor immunity (see, Nature. 2010; 465(227-31). Three weeks later, when a memory response was established against the NMD inhibition-induced neoantigens, mice were challenged subcutaneously with 4T1 breast carcinoma cells that do not cross-react with the CT26 tumor antigens. 7-8 days later when tumors become palpable, neoantigens were induced in the 4T1 tumors by systemic administration of nucleolin aptamer-targeted Smg-1 siRNA. As shown in FIG. 4, only when the CT26 derived vaccine was made in the presence of DOX and only when the 4T1 tumors were targeted with Smg-1, but not control, siRNA, 4T1 tumor growth was significantly inhibited. This experiment supports the hypothesis that neoantigens induced in the CT26 vaccine elicited an immune response that inhibited the growth of 4T1 tumors, provided they were made to express the neoantigens, also suggesting that at least a proportion of the NMD-inhibition neoantigens are shared between CT26 and 4T1 tumors.

Example 4 Induction of Neoantigens in Tumor Cells by Inhibiting Key Mediators of Antigen Processing: TAP and ERAAP

siRNA targeting will employ nucleolin binding aptamers. Nucleolin is also upregulated on the tumor vasculature. An advantage is that if the protective antitumor response (e.g., FIGS. 2-4) is mediated by an endothelial-specific immune response, nucleolin targeting will be near universally effective against all tumors. Previous studies have shown that vaccination against endothelial cells or tumor stromal product not expressed in tumor cells can stimulate effective antitumor immunity in mice in the absence of significant autoimmunity.

A targeting strategy using a 19 nt long EpCAM specific aptamer (e.g., 5′-GCGACUGGUUACCCGGUCG-3′) (SEQ ID NO: 1) is undertaken. The EpCAM aptamer originally isolated for binding to human EpCAM also binds to murine EpCAM. EpCAM, which is weakly expressed on the basolateral gap junction of epithelial cells and is not accessible to drugs, is highly upregulated on most tumor cells of epithelial origin including breast, lung, colon, pancreas, and prostate cancer. Thus targeting EpCAM will be broadly applicable to many tumors of distinct origin, but unlike nucleolin will not bind to tumor vasculature. Both nucleolin and EpCAM targeting will be evaluated for tumor inhibition and (lack of) toxicity.

Pharmacokinetics and tumor and tissue distribution of intravenously injected nucleolin aptamer and its conjugates will be determined in tumor bearing mice using ³²P-labeled conjugates (see Mol Ther. 2011; 19(10):1878-86; Cancer Immunology Research. 2014; 2(9):867-77). Intravenously injected aptamer conjugates exhibit a circulation half-life of about 18-48 hours and accumulate in the tumor rapidly, within 2-6 hours after injection. It is expected that the nucleolin-targeted conjugates will exhibit preferential accumulation in the tumor tissue. Indeed, FIG. 5 shows that intravenously administered ³²P-labeled Nuc-TAP siRNA conjugates home to and accumulate preferentially in the subcutaneously implanted tumor compared to normal tissues, including lung, heart, liver, spleen, kidney and TDLN.

Inhibition of tumor growth in the tamoxifen-induced BRAF mutant melanoma and autochthonous PDA models will be studied. Aptamer-siRNA conjugates will be first screened in the transplantable subcutaneously implanted palpable 4T1 model (FIG. 2) and best-in-class conjugates will be used in the advanced models. It is expected that ERAAP or TAP downregulation will be superior to NMD (Smg-1) downregulation on account of generating mostly shared neoepitope and/or reduced risk of enhancing tumorigenicity and the combination of ERAAP and TAP downregulation will be additive because they generate distinct neoepitope providing an increased neoantigen burden. Selected best-in-class (combinations of) conjugates will be tested in the BRAF mutant melanoma and PDA models.

BRAF mutant melanoma model. The studies use the tamoxifen-induced BRAF/PTEN model Nature Genetics. 2009; 41(5):544-52, FIG. 3). Briefly two strains of mice are mated and F1 mice are induced with hydroxytamoxifen applied to the skin. Tumor develop locally within 3-4 weeks with 100% penetrance and subsequently metastasize to the ear and base of tail in which case they can be followed in real time, as well as the inguinal lymph nodes and lung which can be evaluated post mortem. Treatment with aptamer conjugates injected i.v. starts when the local tumors reach about 3 mm in height and survival will be used as main endpoint.

Autochthonous PDA model. A model of pancreatic cancer generated by surgically implanting 3 mm tumor fragments from KPC mice into the pancreas of wild type C57BL/6 mice (˜10 mice/KPC tumor) is used (Journal of Gastrointestinal Surgery 2016; 20(1):53-65). Tumors develop synchronously recapitulating the intense desmoplasia and leukocytic infiltration seen in the genetically engineered KPC mice. Tumors are histologically detectable after 4 weeks and mice develop morbidity requiring euthanization after about 10-12 weeks. In this model treatment with aptamer conjugates will start week 3-4 after tumor implantation. Progressive weight loss and survival will be used as main endpoints. Only the most effective strategies will be tested in the spontaneous KPC model.

Another model that may be used is the MCA carcinogen-induced fibrosarcoma model (Cancer Immunology Research. 2014; 2(9):867-77).

Neoantigen identification. The dominant shared neoantigens generated by the best-in-class approach(s) discussed above will be identified. Mass spectrometry based immunopeptidomics will be used to identify naturally presented neoepitopes on the surface of cells in which NMD, ERAAP, or TAP were downregulated (Curr Opin Immunol. 2016; 41(9-17)).

Immunological mechanisms underlying the induction of tumor immunity will be tested. The hypotheses that neoantigen expression will (i) increase the intratumoral immune infiltrate that will exhibit an immune stimulatory/inflammatory signature, and that such immune infiltrate will correlate with the potency of the antitumor immune response evaluated in the immunotherapy models, and (ii) induce a potent CD8+ and indirectly CD4+ T cell mediated adaptive immune response when TAP or ERAAP are downregulated will be tested.

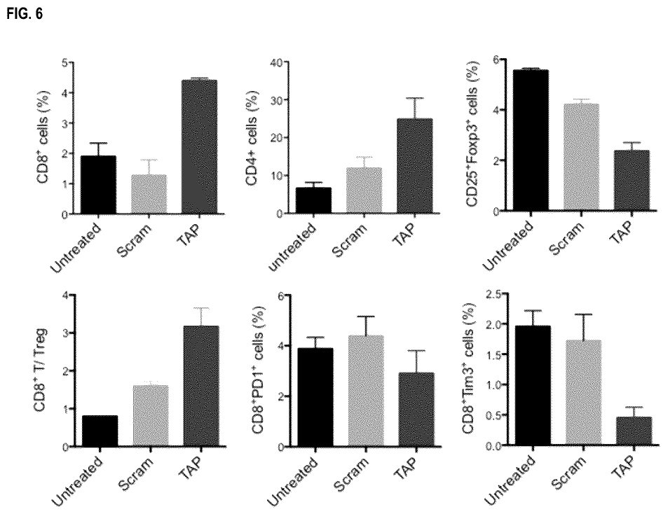

With regard to intratumoral immune infiltration, it will be determined by multiparameter flow cytometry of intratumoral immune infiltrates including but not limited to CD4 and CD8 T cells, Treg (CD4+Foxp3+), tumor cross-presenting DC (CD11c+ Flt3L+CD103+CCR7+), MDSC (CD11b+Gr1+), macrophages (CD11b+F4/80+), tumor resident memory T cells (CD8+CD69+, CD62L-CCR7-), exhausted T cells expressing a combination of PD-1, Tim3, and/or LAG3, and presence of polyfunctional CD4 and CD8 T cells expressing IFN, IL-2 and TNF which correlate with protective immunity. In a preliminary experiment (FIG. 6) it is shown that Nucl-TAP siRNA inhibition of tumor growth was accompanied by an increased CD4+ and CD8+ T cell infiltrates, increase in CD8+ T/Treg ratio, reduction of Treg, and a very significant reduction in deeply exhausted Tim-3+CD8+ T cells, all evidence of a robust antitumor immune response.

The role of adaptive immunity will be tested (a) in nude mice, (b) by antibody depletions of CD4 and/or CD8 subsets, and (c) in vitro proliferative T cell responses with splenic CD4 and CD8 T cells stimulated in vitro with tumor lysate loaded DC. Antigen specific T cell responses against known TEIPPs resulting from TAP or ERAAP downregulation will be determined by in vitro stimulation of splenic CD8 T cells with peptide loaded DC.

Establishment of immunological memory will be evaluated by rechallenging mice that were cured by the treatment with the aptamer-siRNA conjugates. Since it may be difficult to cure mice from palpable 4T1 tumors (see FIG. 2) the more immunogenic A20 or C26 model that can be more readily cured from pre-established palpable tumors (data not shown) will be used.

Contribution of an anti-vasculature immune response will be evaluated. As discussed above, nucleolin is also upregulated on the tumor endothelial cells. Whether an anti-endothelial immune response contributes or is responsible for the observed antitumor response elicited by nucleolin-targeted induction of neoantigens will be evaluated. To that end the cross-protective nature of antitumor immune response, for example whether neoantigen induction mediated inhibition of 4T1 tumors induces protective immunity against CT26 tumors or vice versa will be evaluated This will be done by subsequent contralateral implantation of the second tumor, T cell transfer, or challenge of cured mice. Further, if evidence of cross-protection is observed, to confirm the role of nucleolin targeting it will need to be shown that EpCAM targeting (which is expressed only on tumor cells, not endothelial cells) will not induce cross-protection. If endothelial targeting is primarily responsible for the observed T cell dependent antitumor response it will have important implications in term of the applicability of nucleolin-targeted immune therapy to encompass virtually all cancer patients. However, engendering endothelial specific immune response also increases the risk of autoimmune pathology, which will be evaluated below. If signs of significant toxicity are observed, EpCAM targeting will be further explored.

Toxicity, nonspecific immune activation, autoimmune pathology and enhanced tumorigenicity will be studied as well. No evidence of toxicity in term of morbidity or mortality in mice treated with nucleolin aptamer targeted siRNA conjugates has been observed. In clinical trials administration of ˜100 fold higher doses of the nucleolin aptamer to patients was found to be safe (Exp Mol Pathol. 2009; 86(3):151-64). No toxicity was reported in mice immunized with TAP, ERAAP deficient cells and no toxicity has been observed with tumor targeted NMD inhibition. Nor is nonspecific immune activation anticipated, because the aptamers and the sense strand of the siRNAs contain 2′-fluoro-modified pyrimidines. Toxicity, especially autoimmunity, could however become an issue when using combination treatments, as discussed below. Nonspecific immune activation of the administered conjugates will be assessed by measuring the presence of IFNα, IL-6 and TNF in the circulation. Conjugates that induce nonspecific immune activation will be discarded. This problem has not been encountered so far (over 10 different conjugate tested; three different aptamers). Morbidity and mortality will be inspected visually on a daily basis. Nonspecific inflammation will be evaluated by counting CD4 and CD8+ T cells in the liver, lymph nodes and spleen, and by H&E staining of liver, lung and intestines. Autoimmune pathology will be assessed by measuring liver transaminases in the circulation, AST and ALT. Toxicity that could be associated with an antivasculature immune response will be evaluated by measuring effects of nucleolin targeted immunotherapy on wound healing and pregnancy as described in a previous study (Blood. 2003; 102(3):964-71).

Partial NMD downregulation in stressed cells stabilizes a set of PTC containing mRNAs encoding products that promote cell survival thereby running the risk of enhancing the malignancy of tumor lesions targeted for inhibition of NMD. This is not expected with ERAAP and/or TAP. In a previous study no evidence of NMD inhibition induced enhanced malignancy in nude mice was observed (Nature. 2010; 465 (227-31)). Given that nude mice exhibit robust NK activity this issue will be revisited using a sensitive soft agar colony assay cultured under normoxic as well as hypoxic conditions, the latter mimicking the hypoxic stress of tumor cells in situ. In brief, tumor cells expressing a DOX-regulated NMD shRNA, as well as TAP or ERAAP shRNAs will be plated in soft agar and colony formation, both in terms of rate and number will be measured in presence and absence of DOX. An increase in rate of development and number of colonies formed, that is predicted toll be more pronounced under hypoxic conditions, will be suggestive evidence for enhanced malignancy. Failure to demonstrate increased tumorigenicity will reduce but not eliminate the risks.

FIG. 7 shows that inhibition of NMD enhances the anchorage independent growth of CT26 tumor cells. NMD is partially downregulated in “stressed” cells including tumor cells (Cell Cycle. 2008; 7(13):1916-24). Immune responses generated upon targeted downregulation of NMD in tumor cells could therefore recognize normal cells that are experiencing adverse effect and are under “stress” such as wounds, sites of infection, or tissues experiencing autoimmune sequeala. Of particular concern is that partial NMD downregulation in stressed cells stabilizes a set of premature termination codon (PTC) containing mRNAs encoding products that promote cell survival, like autophagy (Molecular and Cellular Biology. 2013; 33(11):2128-35) or amino acid uptake and biosynthesis (Nature Genetics 2004; 36(10):1073-8), thereby running the risk that tumor targeted inhibition of NMD will full enhance their malignancy (Molecular and Cellular Biology 2011; 31(17):3670-80). In FIG. 7, this concern was tested by simulating stress in tumor cells by growing them in hypoxic conditions, a major source of stress of tumor cells in situ. It was hypothesized that downregulating NMD in tumor cells will enhance their ability to form colonies in soft agar that will be enhanced under stress (i.e. hypoxic) conditions. NMD was downregulated in a controlled manner by stably expressing an Smg-1 shRNA under the control of doxycyline (Nature. 2010; 465(227-31). The experiment shows that whereas under normoxic conditions (20% O₂) the colony forming potential of NMD inhibited (+DOX) tumor cells was only marginally superior, under hypoxic conditions (one week in 0.5% O₂ followed by one week of 20% O₂), the colony forming potential of the NMD inhibited tumor cells was significantly superior to that of the NMD sufficient (−DOX) tumor cells. Arguably, and without wishing to be bound by theory, FIG. 7 reinforces the concern that tumor targeted NMD inhibition in tumor cells in vivo could enhance their malignancy.

### Example 5: Combination Strategies to Potentiate the Neoantigen Induced Antitumor Immune Response

Evaluation of combination therapies of neoantigen induction and immune potentiating therapies will be undertaken with a goal of identifying complementary strategies that will enhance the antitumor response generated as a result of expressing neoantigens in the tumor cells. Combination strategies will be screened in the 4T1 model (FIG. 2) and selected combinations will be evaluated in the BRAF mutant melanoma and autochthonous PDA models.

Tumor lesions are poorly infiltrated by proinflammatory immune cells, which is a main reason why they are not optimally responsive to checkpoint blockade therapy, and conceivably other forms of immune potentiating therapies. Neoantigen expression alone is not sufficient to promote immune infiltration. It was recently shown that one mechanism preventing the intratumoral trafficking of immune cells is mediated by the wnt/β-catenin pathway, and that absence of p-catenin expression in tumor cells converts “noninflamed” into “inflamed” tumors. 4T1 breast carcinoma and B16.F10 melanoma cells express elevated levels of 1-catenin and its downstream mediator TCF7, 5-8-fold higher level than syngeneic adherent splenocytes or contact inhibited NIH 3T3 cells as determined by qRT-PCR (data not shown). Accordingly, the hypothesis that that tumor targeted downregulation of β-catenin in 4T1 tumors in situ using nucleolin or EpCAM aptamer-siRNA conjugates will enhance intratumoral T cell infiltration and synergize with neoantigen induction to inhibit tumor growth will be tested. An alternative target is PI3Kβ, which can be inhibited with a selective small molecule inhibitor, GSK2636771, or with nucleolin aptamer targeted siRNAs.

Other methods to promote intratumoral immune infiltration that will be considered are local irradiation or intratumoral administration of STING ligand.

Further, combination with checkpoint blockade with CTLA-4 and PD-1 antibodies will be evaluated. Checkpoint blockade with CTLA-4 and PD-1 antibodies to counter the function of inhibitory receptors expressed on tumor infiltrating T cells is arguably the flagship of cancer immunotherapy. Despite unprecedented clinical responses as monotherapy it is not a cure and a significant fraction of patients do not respond, and in the case of CTLA-4 therapy can exhibit significant toxicity. The proposed combination studies therefore will monitor toxicities, especially in mice co-treated with CTLA-4 antibodies (see below), including enterocolitis, inflammation of the intestine, the main severe toxicity seen in patients treated with CTLA-4 antibody (e.g., ipilumimab). The toxicities seen with CTLA-4 antibodies in mice have been recapitulated, both as monotherapy or in combination with tumor radiation, characterized by significant inflammatory responses in the intestine, lung and liver, with histological evidence of tissue damage in the intestine.

### Example 6: Strategies to Control Tumor Recurrence and Progression of Precancerous Lesions

Strategies to control tumor recurrence and progression of precancerous lesions are undertaken. First methods to vaccinate against neoantigens with the goal of establishing a potent and long lasting neoantigen-specific immune response are established, and then combinations of vaccination and neoantigen induction are tested, e.g., as shown in FIG. 1, using murine models for recurrent disease and models that recapitulate the cancer development process.

Eliciting an immune response against the neoantigens by vaccination will obviate the reliance on the endogenous immune response against the tumor-induced neoantigens within the immune suppressive tumor microenvironment. The underlying premise, without wishing to be bound by theory, is that the neoantigens used in the vaccine and subsequently induced in the tumors are the same.

Any of the following approaches may be used to generate the vaccine response:
Vaccination with lysate loaded ex vivo generated DC, whereby the lysate was generated from the subject's normal tissue in which one or more of ERAAP, TAP, and Ii is downregulated either by nucleolin-siRNA or by shRNA expressing lentiviral vectors. Sources of normal tissue can be fibroblasts or B cells that can be readily expanded in vitro in short term cultures. Instead of lysate, it would be possible to use RNA from the tumor, total or mRNA enriched poly A+ RNA. Poly A+ RNA can be also amplified to generate sufficient antigen for DC loading and thereby limit the ex vivo culture step
Vaccination with neoantigen mRNA-lipid nanocarriers. Vaccination with mRNA complexed to standard lipid carriers like DOPE and DOTMA can be undertaken (Nature. 2016; 534(7607):396-401). Vaccination with mRNA-lipid complexes exhibiting a net positive charge has been previously used but was not particularly effective. Tweaking the net charge of the RNA to lipid ratio to be slightly negative led to the targeted accumulation and uptake of the systemically administered complexes by antigen presenting cells in the spleen and lymph node and generation of immune response of unprecedented magnitude in mouse immunotherapy models and in preliminary studies in human patients (Nature. 2016; 534(7607):396-401). This approach will be used to vaccinate against neoantigens using total RNA, mRNA enriched poly A+RNA, or amplified polyA+RNA from syngeneic fibroblasts or B cells as described above.
Inducing neoantigens in DC in situ. Expression of the neoantigens in the DC in situ will be undertaken. The approach will be to target the neoantigen inducing siRNA (to inhibit one or more of ERAAP, TAP, and Ii) to DC by conjugating the siRNAs to a DEC205 aptamer or a TLR9 stimulating CpG oligonucleotide (ODN). DEC205 is a lectin-like receptor expressed on immature DC that is responsible for the uptake and cross-presentation of apoptotic cells to both CD4+ and CD8+ T cells. DEC205 conjugated antigens stimulate potent T cell responses in mice, provided a DC maturation agent is included in the protocol like CD40 antibody, poly I:C or CpG. A DEC205 aptamer that was shown to target the OVA antigen to DC in vitro and in vivo will be used. DEC205-siRNA conjugates will be characterized in vitro for DEC205 dependent downregulation of their corresponding targets in DC and the consequences on their functionality, namely improved stimulation of antigen-specific T cell responses. Validated DEC205 aptamer-siRNA conjugates will be used in mouse immunotherapy experiments by administration into the circulation via tail vein injection together with the well characterized 1680 phosphorothioate CpG ODN. Conditions in terms of regimen, dose, or alternative adjuvants like poly I:C, will be evaluated using DEC205-ERAAP siRNA and measuring the induction of CD8+ T cell responses against a defined ERAAP deficient-induced epitope, the Qa-1b restricted FYAEATPML (FL9) peptide derived from FAM49B protein. Alternatively, the siRNA will be targeted by conjugation to a CpG ODN. CpG ODNs have been successfully used to target STAT3 siRNA to DC in situ. An important advantage of CpG ODN targeting is that it will dispense with the need of providing separately a DC maturation signal. The chemically synthesized DEC205 or CpG-siRNA conjugates would, therefore, represent a universally applicable cost-effective drug to induce immunity against said neoantigens in both prophylactic and therapeutic settings. Another option that will be explored is to co-deliver unconjugated siRNAs and CpG ODN or Poly I:C as DC maturation agents to DC in situ by encapsulation in the anionic lipoplexes discussed above.

Preventing and treating disease recurrence will be tested in two models for recurrent cancer: post-surgical metastasis model for breast cancer and post-surgical local recurrence model for pancreatic cancer. In the post-surgical metastasis model for breast cancer model (Cancer Immunology Research. 2014; 2(9):867-77) 4T1 breast carcinoma tumor cells are implanted in the abdominal fat pad, 9-11 days later when tumors become readily palpable they are surgically excised at which time the mice are vaccinated. Tumors recur locally in about 50% of mice due to incomplete resection, the equivalent of the clinical phenomenon known as “positive margins”, and lung metastases develop 4-5 weeks later at which time lung are evaluated for metastatic burden, or mice are monitored for survival. In the post-surgical local recurrence model for pancreatic cancer model (Journal of Gastrointestinal Surgery 2016; 20(1):53-65) the KPC derived surgically implanted tumors in the pancreas are surgically resected at which time mice are vaccinated. Tumors recur locally 3-5 week later due to incomplete resection.

Exploiting the ability induce neoantigens in tumor cells in situ, development of a new paradigm for prophylactic, though not preventative, cancer vaccination as discussed, inter alia, is FIG. 1, panel B, whereby healthy individuals, at risk for developing cancer, are vaccinated prophylactically against neoantigens and if or when tumor develops, the same neoantigens are induced in the patient’ tumor (“Prorapeutic” immunotherapy).

Protocols will be first tested in prophylactic settings using the transplantable 4T1 model as shown in FIG. 4, and similarly autochthonous PDA model whereby nontumor bearing mice are vaccinated against neoantigens and subsequently challenged with the KPC-derived tumor fragments. To simulate individuals at risk two models with precancerous lesions that will progress over several months to malignant tumors will be used:
The Balb-neuT transgenic model for breast carcinoma. Balb-neuT mice carry an activated rat Her2/neu oncogene expressed under the transcriptional control of a long terminal repeat of a mammary tumor virus. All offspring develop mammary carcinoma at high multiplicity after a latency of 4-5 month. Tumor progression in the Balb-neuT mice recapitulates the process and stages in breast cancer development in human patients in a highly synchronous fashion, atypical hyperplasia (week 6), in situ carcinoma (weeks 14-16) to invasive lobular carcinoma that can be detected histologically in all mice 6 to 7 month of age (reviewed in Cancer Immunol Immunother. 2004; 53(3):204; Breast Dis. 2004; 20(33-42).
The MCA-induced fibrosarcoma carcinogenesis model. MCA-induced tumors develop slowly recapitulating the multistep carcinogenesis process, becoming palpable in about 70-90 days. MCA-induced tumors are, however, nonmetastatic. In this well-established broadly used model (see for example Nat Med. 2006; 12(6):693-8) mice are injected subcutaneously with 200-400 μg MCA, tumors becoming palpable around weeks 10-12. In the absence of treatment tumors reach maximum allowable size at weeks 25-30.

In both models neoantigen vaccination will be carried out at the premalignant stage and when tumors become palpable systemically administered with nucleolin or EpCAM aptamer-ERAAP or TAP conjugate and if so indicated an additional therapeutic agent.

In the setting of prophylactic vaccination against neoantigens expressed in a future tumor that may arise month to years later, long term persistence of vaccine-induced immune response is important. To that end an approach to inhibit mediators of CD8+ T effector cell differentiation in vivo that skews the differentiation of activated T cells to become long lasting memory cells has been developed. It has been shown that systemic administration of 4-1BB aptamer targeted raptor siRNA to mice downregulated mTORC1 function in the majority of circulating vaccine-activated CD8+ T cell (mTORC1 is a key mediator of effector functions in T cells), leading to the generation of potent memory responses, and enhanced protective antitumor immunity in tumor bearing mice, and the 4-1BB aptamer siRNA downregulation CD25 or Axin-1 also potentiate vaccine-induced tumor immunity. Whether combination therapy with neoantigen vaccination will enhance the persistence of protective immunity against (neoantigen-engineered) tumors that will develop many months after vaccination will be tested.

### Example 7: Neoantigen Induction by Invariant Chain (Ii) Downregulation

TAP and ERAAP are components of the MHC class I presentation pathway and therefore their downregulation will promote the generation of class I restricted CD8 T cell responses. MHC class ∥ restricted CD4+ T cell responses are, however, also important in the setting of tumor immunity (J Exp Med. 1999; 189(5):753-6; Curr Opin Immunol. 1998; 10(5):588-94), underscored by recent clinical trials targeting the CD4+ T cell arm of the antitumor immune response (Nature. 2015; 520(7549):692-6; Science. 2014; 344(6184):641-5). Expression of the MHC class II presentation machinery that includes the classical MHC genes HLA-DP, DQ and DR, Invariant chain, and the nonclassical regulatory proteins HLA-DM and DO, are regulated by the inflammation and IFNg-inducible master transcription coactivator CIITA (Nat Rev Immunol. 2011; 11(12):823-36). The MHC class II locus can be induced by treatment with IFNg or with demethylating agents like HDAC inhibitors (Immunol Res. 2010; 46(1-3):45-58). Most tumor cells except for B cell derived tumors do not express MHC class II. In many but not all tumors class II expression can be induced with IFNg or HDAC inhibitors (Clin Immunol. 2003; 109(1):46-52; Br J Cancer. 2000; 83(9):1192-201). Conceivably such tumor also upregulates class II expression in situ, especially following vaccination that generates an inflammatory environment. Whereas MHC class II molecules can bind endogenously derived peptides originating from the cell membrane or from other cellular compartments via an autophagocytic process (rontiers in immunology. 2012; 3(9)), the nascent class II molecules associate preferentially with exogenously derived peptide that is regulated by the Invariant chain to disfavor the binding of endogenously derived peptides (Rev Immunol. 2011; 11(12):823-36). However, in the absence of Invariant chain class II binding of endogenous peptides is significantly enhanced (Immunol. 1994; 153(4):1487-94; Science. 1994; 263(5151):1284-6; Eur J Immunol. 1994; 24(7):1632-9; Proc Natl Acad Sci USA. 1997; 94(13):6886-91). Since such endogenous peptides are normally not presented in the Ii+ cells, they could function as neoantigens.

This was exploited to enable class II negative tumors to stimulate CD4 T cell responses against endogenous class II restricted antigens and/or sensitize them to CD4 T cells by either cotransfecting of tumor cells with the two chain comprising a class II molecule in the absence of Ii (J Immunol. 1990; 144(10):4068-71), or by treating tumor cells with IFNg or transfected with CIITA to upregulate the MHC locus and then incubated with Ii antisense RNA to downregulate Ii (Cancer Immunol Immunother. 1999; 48(9):499-506). Treatment of mRNA transfected DC with Ii antisense RNA promotes the class II presentation of the cytoplasmically expressed mRNA encoded antigen leads to enhanced class II presentation, CD4 T cell response and improved antitumor immunity (Blood. 2003; 102(12):4137-420).

This Example shows that downregulation of Ii in tumor cells in situ represents an approach to induce class II restricted neoantigens and generate CD4 T cell responses, that could synergize with the generation of class I restricted CD8+ T cell response by downregulation of ERAAP or TAP.

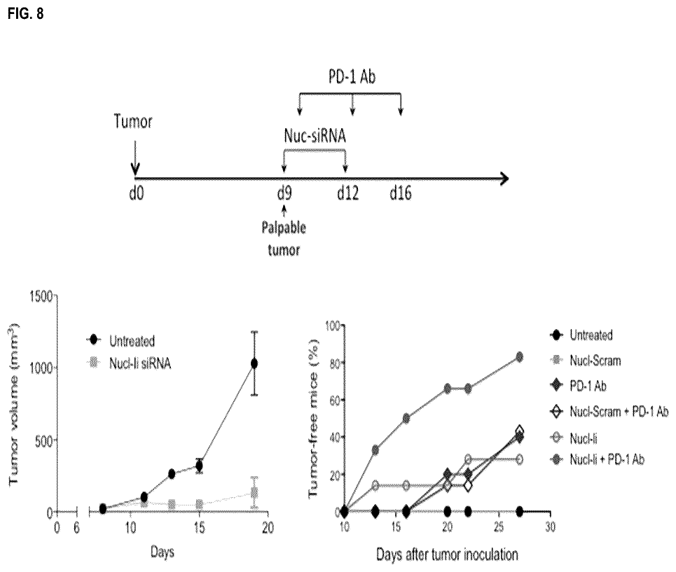

To test whether tumor-targeted Ii downregulation can inhibit tumor growth 4T1 tumor bearing mice were treated with nuclelin aptamer-Ii siRNA (Nucl-Ii siRNA) conjugates and tumor growth monitored by measuring tumor volume (left panel of FIG. 8) or regression of the implanted and palpable tumors (right panel of FIG. 8). As shown in FIG. 8, treatment of the tumor bearing mice with Nucl-Ii siRNA inhibited tumor growth leading to tumor regression in a proportion of mice, and enhanced the antitumor effect of PD-1 Ab blockade (right panel of FIG. 8).

Tumor growth was significantly inhibited in mice treated with Nucl-Ii siRNA measured as tumor volume (bottom left panel of FIG. 8) or tumor-free mice experiencing regression of the established tumor (bottom right panel of FIG. 8).

In summary, and without limitation, downregulation of specific mediators of antigen processing pathway like TAP, ERAAP or Invariant chain present novel epitopes to which the immune system has not been tolerized and thereby they could function essentially as neoantigens. Such epitopes are nonmutated subdominant epitopes that are normally not presented and therfore unlike the NMD-inhibition epitopes carry a reduced risk of autoimmunity. Importantly, epitopes generated by downregulation of TAP, ERAAP or II are not generated as a result of random events in the cell therefore they are more like to be shared, namely the same epitope presented any cell in which the corresponding target was downregulated. This represents another potential advantage over the NMD inhibition approach.

### Example 8: Prorapeutic Immunotherapy

This example further tests the “prorapeutic immunotherapy” approach shown, by way of non-limiting illustration, in FIG. 1, and follows up on the proof-of-concept experiment shown in FIG. 4. In the experiment shown in FIG. 4 the vaccination step to induce neoantigens was done by using irradiated tumor cells engineered to express neoantigens by stable downregulation of NMD using Smg-1 shRNA. The clinical translatability of this approach may be cumbersome (one would need to take autologous tumor cells on a patient-by-patient basis, and then further manipulate them in vitro, etc.). This example explores a clinically more useful vaccination protocol with a small chemically synthesized reagent one-drug-fits-all (cancers), injected in the patient.

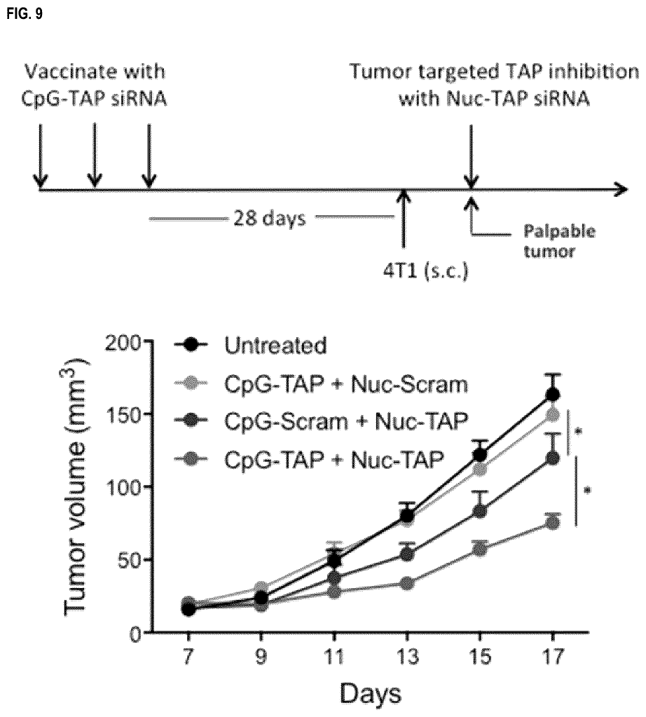

siRNAs used to induce neoantigen—TAP siRNA used in this experiment for illustration—was targeted to dendritic cells in situ by conjugation to a short CpG olignucleotide. Previous studies have shown that CpG ODNs can target siRNAs or short DNA ODNs to DC in situ (Nat Biotechnol 27: 925-932). CpG ODNs are beneficial because they not only target the attached cargo to DC (via binding to TLR9) but also activate/mature the DC which leads to induction of immunity against the TAP knockdown induced neoantigens as opposed to tolerance. The experiment shown in FIG. 9 shows prophylactic vaccination against future tumors with CpG-TAP siRNAs was successful.

### Example 9: HLA-E Restricted Neoepitopes Induced in Cells in which ERAAP or TAP are Downregulated

Normally class I restricted T cell epitopes are presented by the polymorphic HLA-A, B, or C alleles, but not the nonpolymorphic HLA-A allele. However, when ERAAP or TAP are downregulated, inter alia, a significant proportion of the neoepitopes/neopeptides are presented by the monomorphic HLA-A allele (known as Qa-1 allele in mice) (REF). Given that such neoepitopes are encoded in non-mutated house-keeping products expressed in mostly all tumor cells, most HLA-E restricted neoepitopes are common to all tumor cells in which ERAAP or TAP, and most likely, other mediators of class I antigen processing, are downregulated, regardless of the haplotype of the cells, which is dictated, inter alia, by the canonical polymorphic alleles. Accordingly, a single TCR or CAR will recognize every tumor cell in which said mediators of antigen processing is downregulated. This results in applications including a universal vaccine consisting of such epitopes in the form of peptides, RNA, whole protein, and/or DNA, a universal immune monitoring system for (vaccinated) patients for T cell responses against TAP, ERAAP downregulation-induced neoantigens, for example using HLA-E/neopitope tetramers and a universal adoptive T cell therapy approach, one or more of a mixture of several, including, universal TCRs or CARs that will be transduced in the any patient's T-cells and said mediators, i.e., ERAAP or TAP, downregulated in the patient's tumor by targeted delivery of corresponding siRNA. An important advantage is that expression and presentation of the neoepitopes and thereby stimulation of the TCR or CAR expressing T cells will be transient, primarily because it is controlled by aptamer-targeted siRNA inhibition which is transient, and thereby reduces concerns of T cell dysfunction or toxicity.

### Preventing Recurrence

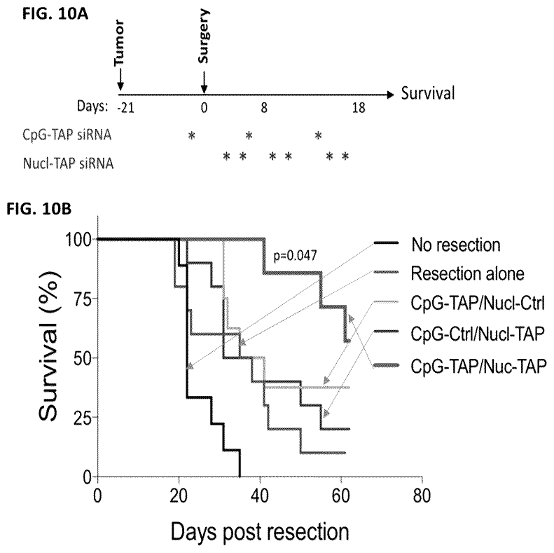

To examine a model for remission/MRD (minimal residual disease) for preventing recurrence which is a challenge in clinical oncology, patients in remission against the induced neoantigens are vaccinated and following the recurrence of the tumors, the antigens are induced. As shown in FIG. 10A, pancreatic cancer cell line model (KPC cells) was mixed with pancreatic fibroblasts (stellate cells) and surgically implanted in the pancreas of the mouse, closely recapitulating the unique biology and resistance to treatment of pancreatic cancer in humans (REF). When tumors became palpable they are surgically resected and recurrence was followed by measuring survival. Mice are vaccinated against the induced neoantigens (CpG-TAP) and said neoantigens are induced in the recurring tumors (Nucl-TAP). Experiment shows that when vaccination is combined with induction, there is a significant inhibition of recurrence/extension of survival (FIG. 10B).

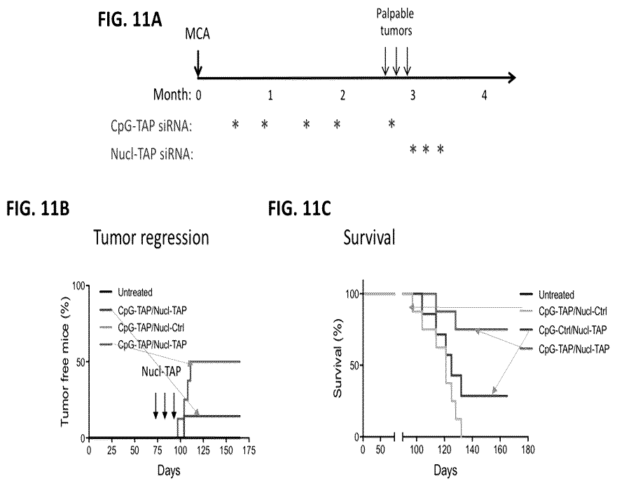

To examine a model of premalignancy for preventing future tumor development, patients at risk of developing cancer, patients with premalignant lesions, chronic infections or genetic predisposition are vaccinated against induced neoantigens, and if a tumor develops induce the said neoantigens in the developing tumor. As shown in FIG. 11A, experimentally, carcinogen-induced model for fibrosarcoma whereby mice are first treated with carcinogen, methyl cholanthrene (MCA) and tumor develop about three months later. Mice are vaccinated against the induced neoantigens (CpG-TAP) and when tumors develop said antigens are induced in the developing tumors (Nucl-TAP). As above, when vaccination is combined with induction, a significant therapeutic impact in terms of complete tumor regression in 50% of the mice (FIG. 11B) and majority of mice surviving long-term including mice with small tumors that do not continue to grow (FIG. 11C).

### A Single Agent/Drug to Vaccinate and Induce Neoantigens in Tumors for B Cell Malignancies

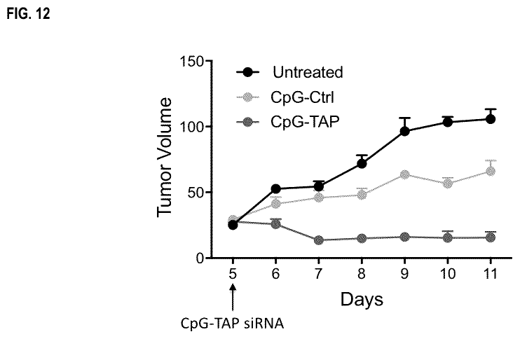

Most B cell malignancies (like dendritic cells) also express TLR-9, the endocytic receptor for CpG. Thus the CpG oligonucleotide targets the (TAP, ERAAP) siRNA to both DC as well as tumor. This system does not require a second tumor-targeting agent like nucleoling (Nucl). As shown in FIG. 12, a single injection of CpG-TAP siRNA to day 5 tumor bearing mice, mice implanted with the TLR9-expressing A20 B cell lymphoma tumor prevented tumor development.

### Vaccination Against Existing, Concurrent Tumor: Therapeutic Vaccination

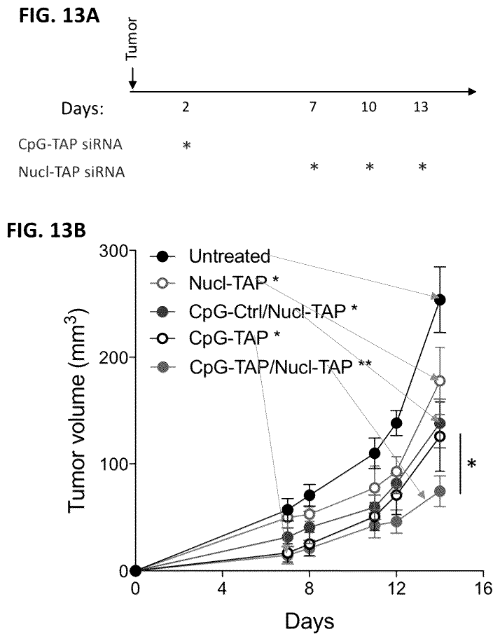

Unlike the condition where patients in remission to prevent recurrence, or individuals against a future tumor are vaccinated, in therapeutic vaccination, patients with existing measurable disease, such as patients that cannot be induced into remission, or in the neoadjuvant setting before debulking are vaccinated. As shown in FIG. 13A. mice were vaccinated against the induced neoantigens (CpG-TAP) and said neoantigens were induced in the recurring tumors (Nucl-TAP). Cancer vaccination against existing, concurrent tumor resulted in a decrease in tumor volume at days 4, 8, and 12 following injection of CpG-TAP siRNA when compared to untreated, Nucl-TAP, CpG-Ctrl/Nucl-TAP, CpG-TAP and CpG-TAP/Nucl-TAP (FIG. 13B).

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

What is claimed is:

1. A method of treating cancer in a subject need thereof, comprising administering an effective amount of an immune-modulating agent to the subject's cancer cells to direct a subject's existing immune response to a neoantigen against the cancer, wherein:
the immune-modulating agent inhibits and/or downregulates a mediator of antigen processing and induces neoantigen formation; and
the subject has an existing immune response against the induced neoantigen.

2. The method of claim 1, wherein the method reduces the likelihood of developing the cancer.

3. The method of claim 1 or 2, wherein the subject has previously been afflicted with a cancer.

4. The method of any of claims 1-3, wherein the subject is characterized by one or more of a high risk for a cancer, a genetic predisposition to a cancer, a previous episode of a cancer, a family history of a cancer, and exposure to a cancer-inducing agent.

5. The method of any of claims 1-4, wherein the immune-modulating agent elicits and/or boosts an anti-tumor immune response.

6. The method of any of claims 1-5, wherein the immune-modulating agent inhibits and/or downregulates a mediator of an antigen processing pathway.

7. The method of any of claims 1-6, wherein the immune-modulating agent inhibits and/or downregulates one or more of a mediator of ERAAP, transporter associated with antigen processing (TAP), and invariant chain (Ii).

8. The method of any of claims 1-7, wherein the immune-modulating agent comprises an oligonucleotide molecule, such as a small interfering RNA, or a micro RNA, or an antisense RNA directed against the mediator of antigen processing or a gene-editing protein directed against the mediator of antigen processing, the gene-editing protein selected from a Clustered Regularly Interspaced Short Palindromic Repeats (CRISPR), TALEN, ncikase, and zinc finger protein.

9. The method of any of claims 1-8, wherein the immune-modulating agent further comprises a targeting agent.

10. The method of claim 9, wherein the targeting agent is oligonucleotide aptamer ligand or a protein-based targeting agent.

11. The method of any of claims 1-10, wherein the immune-modulating agent is targeted to a dendritic cell of a subject.

12. The method of claim 11, wherein the dendritic cell is loaded ex vivo.

13. The method of claim 11, wherein neoantigens are induced in DC in situ (optionally via one or more of CpG, DEC205, and CD40).

14. The method of any of claims 1-13, wherein the immune-modulating agent is delivered to the subject via a lipid carrier.

15. The method of any of claims 1-14, further comprising administering an additional therapeutic agent.

16. A method for treating or preventing a cancer in a subject comprising administering, in order:
(a) a therapeutically effective amount of the immune-modulating agent to said subject in need of such treatment, wherein the human subject has developed or is susceptible to developing cancer and wherein the immune-modulating agent stimulates a neoantigen-directed immune response in the subject, and
(b) a different immune-modulating agent than step (a) to the subject's tumor to stimulate the same neoantigens as step (a) and direct the subject's neoantigen-directed immune response against the tumor.

17. The method of claim 16, wherein the subject has previously been afflicted with a cancer.

18. The method of claim 16 or 17, wherein the subject is characterized by one or more of a high risk for a cancer, a genetic predisposition to a cancer, a previous episode of a cancer, a family history of a cancer, and exposure to a cancer-inducing agent.

19. The method of any of claims 16-18, wherein the immune-modulating agent elicits and/or boosts an anti-tumor immune response.

20. The method of any of claims 16-19, wherein the immune-modulating agent inhibits and/or downregulates a mediator of an antigen processing pathway.

21. The method of any of claims 16-20 wherein the immune-modulating agent inhibits and/or downregulates one or more of a mediator of ERAAP, transporter associated with antigen processing (TAP), and invariant chain (li).

22. The method of any of claims 16-21, wherein the immune-modulating agent comprises an oligonucleotide molecule, such as a small interfering RNA, or a micro RNA, or an antisense RNA directed against the mediator of antigen processing or a gene-editing protein directed against the mediator of antigen processing, the gene-editing protein selected from a Clustered Regularly Interspaced Short Palindromic Repeats (CRISPR), TALEN, ncikase, and zinc finger protein.

23. The method of any of claims 16-22, wherein the immune-modulating agent further comprises a targeting agent.

24. The method of claim 23, wherein the targeting agent is oligonucleotide aptamer ligand or a protein-based targeting agent.

25. The method of any of claims 16-24, wherein the immune-modulating agent is targeted to a dendritic cell of a subject.

26. The method of claim 25, wherein the dendritic cell is loaded ex vivo.

27. The method of claim 25, wherein neoantigens are induced in DC in situ (optionally via one or more of CpG, DEC205, and CD40).

28. The method of any of claims 16-27, wherein the immune-modulating agent is delivered to the subject via a lipid carrier.

29. The method of any of claims 16-28, further comprising administering an additional therapeutic agent.


SEQUENCE LISTING (2 sequences)

SEQ ID NO: 1 - RNA, 19 nt, synthetic construct
  source 1..19: mol_type = other RNA
gcgactggttacccggtcg

SEQ ID NO: 2 - protein, 27 aa, synthetic construct
PHETYRALAGLUALATHRPROMETLEU